(19) United States
(12) Patent Application Publication
(10) Pub. No.: US 2025/0297149 A1
(43) Pub. Date: Sep. 25, 2025

(54) UNDERWATER ADHESIVE

(71) Applicant: The University of Tokyo, Tokyo (JP)

(72) Inventors: Takuzo AIDA, Tokyo (JP); Yoshimitsu ITOH, Tokyo (JP); Kohei KIKKAWA, Tokyo (JP); Yu YANAGISAWA, Tokyo (JP)

(73) Assignee: The University of Tokyo, Tokyo (JP)

(21) Appl. No.: 18/842,174

(22) PCT Filed: Mar. 2, 2023
(86) PCT No.: PCT/JP2023/007727

§ 371 Date: Aug. 28, 2024

(30) Foreign Application Priority Data
Mar. 2, 2022 (JP) 2022-032198

(51) Int. Cl. C09J 181/02; C07C 335/10; C07C 335/12; C07C 335/14; C08G 75/045; C09J 5/00

(52) U.S. Cl. CPC C09J 181/02; C07C 335/10; C07C 335/12; C07C 335/14; C08G 75/045; C09J 5/00; C07C 2601/14

(57) ABSTRACT

Provided is a compound, which is represented by the following formula (1):

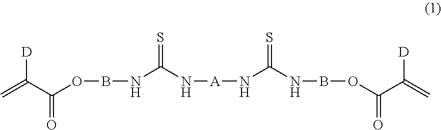

in the formula (1), A and B satisfy a relationship (I) or (II):
(I) one of A or B represents a group represented by the following formula (2); and (II) both of A and B each independently represent a group represented by the following formula (2):

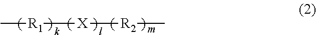

and D represents hydrogen or a methyl group.

### TECHNICAL FIELD

The present invention relates to an underwater adhesive.

### BACKGROUND ART

The number of underwater adhesives that may be used in water is not large. A related-art underwater adhesive has at least one of such drawbacks as described below: the adhesive has a low dry adhesive strength; the adhesive has a low underwater adhesive strength; it takes a long time to cure the adhesive; the adhesive needs to be melted and heated for bonding; and a solvent that dissolves the adhesive is required.

The inventors of the present invention have succeeded in synthesizing a polymer compound, which is obtained by polymerizing an ether having amino groups at both of its terminals and thiocarbonyldiimidazole, and a polymer compound, which is obtained by polymerizing an ether having amino groups at both of its terminals and an ether having isothiocyanate groups at both of its terminals (Patent Literature 1). An adhesive including such compound may be used under a dry state or in water.

### CITATION LIST

### Patent Literature

PTL 1: JP 6574259 B2

### SUMMARY OF INVENTION

### Technical Problem

When bonding is performed with the adhesive of Patent Literature 1, the following needs to be performed: the adhesive is brought into contact with the bonding surface of a targetto be fixed thereto; and then the fixed adhesive is melted by heating or is welded by ultrasonic treatment, followed by cooling. In other words, in Patent Literature 1, it is difficult to heat the adhesive alone in water or to apply an ultrasonic wave to the adhesive in the water.

An object of the present invention is to provide a compound, which can be rapidly cured in water and has a high underwater adhesive property, and a polymer thereof.

### Solution to Problem

The present invention comprehends embodiments described below.
Item 1. A compound, which is represented by the following formula (1):

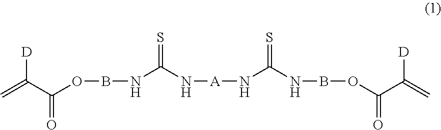

in the formula (1), A and B satisfy a relationship (I) or (II):
(I) one of A or B represents a group represented by the following formula (2):

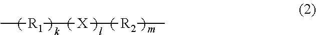

in the formula (2),
R₁ and R₂ each independently represent a substituted or unsubstituted alkylene group having 1 to 16 carbon atoms,
X is selected from the following groups:

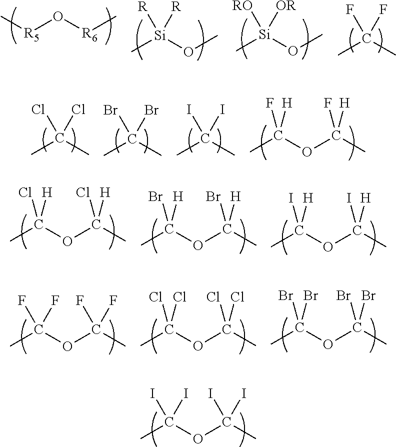

R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “I” represents an integer of 1 or more, and “m” represents an integer of 0 or more,
another of A or B represents one group selected from the following (a) to (c), or has a structure in which the following two or more groups (b) are linked to each other, or a structure in which the following two groups (c) are linked to each other:
(a) a substituted or unsubstituted alkylene group having 1 to 16 carbon atoms; (b):

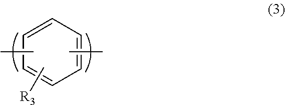

when one bonding moiety of main chain bonding moieties is defined as a 1-position, another bonding moiety thereof may be any one of a 2-position, a 3-position, and a 4-position, and R₃ represents a hydrogen atom, or 1 to 4 monovalent, identical or different, and substituted or unsubstituted alkyl groups each having 1 to 6 carbon atoms; and
(c):

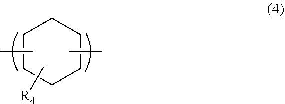

when one bonding moiety of main chain bonding moieties is defined as a 1-position, another bonding moiety thereof may be any one of a 2-position, a 3-position, and a 4-position, and R₄ represents a hydrogen atom, or 1 to 4 monovalent, identical or different, and substituted or unsubstituted alkyl groups each having 1 to 6 carbon atoms, and
when the other of A or B has the structure in which the following two or more groups (b) are linked to each other, or the structure in which the following two groups (c) are linked to each other, a linking group is —O— or —C(Rₐ)(R_{b})—, and Rₐ and R_{b} each independently represent hydrogen or an alkyl group having 1 to 6 carbon atoms; and
(II) both of A and B each independently represent a group represented by the following formula (2):

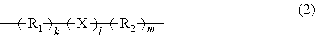

in the formula (2),
R₁ and R₂ each independently represent a substituted or unsubstituted alkylene group having 1 to 16 carbon atoms,
X is selected from the following groups:

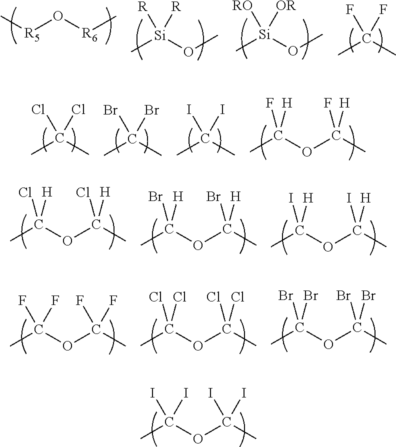

R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “I” represents an integer of 1 or more, and “m” represents an integer of 0 or more, and
D represents hydrogen or a methyl group.
Item 2. The compound according to Item 1, wherein the compound is obtained by reacting a compound represented by the following formula (9) with a compound represented by the following formula (10):

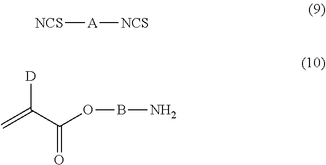

A in the formula (9) and B in the formula (10) satisfy a relationship (I) or (II): (I) one of A or B represents a group represented by the following formula (2):

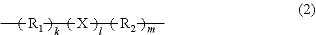

in the formula (2),
R₁ and R₂ each independently represent a substituted or unsubstituted alkylene group having 1 to 16 carbon atoms,
X is selected from the following groups:

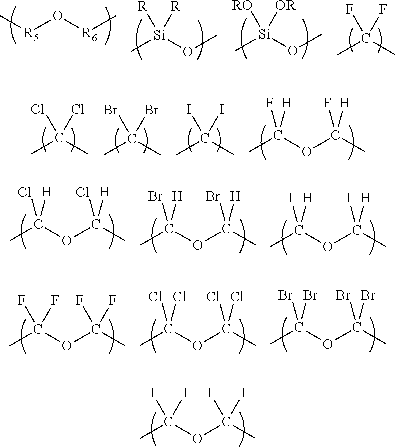

R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “I” represents an integer of 1 or more, and “m” represents an integer of 0 or more,
another of A or B represents one group selected from the following (a) to (c), or has a structure in which the following two or more groups (b) are linked to each other, or a structure in which the following two groups (c) are linked to each other:
(a) a substituted or unsubstituted alkylene group having 1 to 16 carbon atoms; (b):

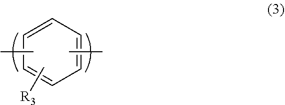

when one bonding moiety of main chain bonding moieties is defined as a 1-position, another bonding moiety thereof may be any one of a 2-position, a 3-position, and a 4-position, and R₃ represents a hydrogen atom, or 1 to 4 monovalent, identical or different, and substituted or unsubstituted alkyl groups each having 1 to 6 carbon atoms; and
(c):

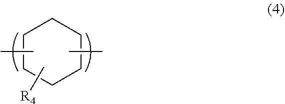

when one bonding moiety of main chain bonding moieties is defined as a 1-position, another bonding moiety thereof may be any one of a 2-position, a 3-position, and a 4-position, and R₄ represents a hydrogen atom, or 1 to 4 monovalent, identical or different, and substituted or unsubstituted alkyl groups each having 1 to 6 carbon atoms, and
when the other of A or B has the structure in which the following two or more groups (b) are linked to each other, or the structure in which the following two groups (c) are linked to each other, a linking group is —O— or —C(Rₐ)(R_{b})—, and Rₐ and R_{b} each independently represent hydrogen or an alkyl group having 1 to 6 carbon atoms; and
(II) both of A and B each independently represent a group represented by the following formula (2):

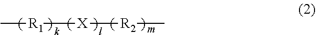

in the formula (2),
R₁ and R₂ each independently represent a substituted or unsubstituted alkylene group having 1 to 16 carbon atoms,
X is selected from the following groups:

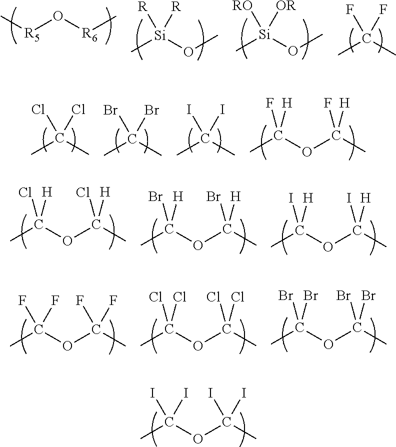

R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “I” represents an integer of 1 or more, and “m” represents an integer of 0 or more, and
D represents hydrogen or a methyl group.
Item 3. The compound according to Item 1 or 2, wherein the group represented by the formula (2) is a group represented by the following formula (2a):

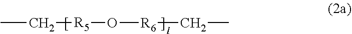

where R₅ and R₆ each independently represent a substituted or unsubstituted alkylene group having 1 to 16 carbon atoms, and a substituent of each of R₅ and R₆ is a linear or branched alkyl group having 1 to 6 carbon atoms, a cyclohexyl group, a phenyl group, or a halogen, and
“I” represents an integer of from 1 to 5.
Item 4. The compound according to Item 1 or 2,
wherein A represents a group represented by the formula (2a):

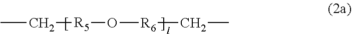

where R₅ and R₆ each independently represent a substituted or unsubstituted alkylene group having 1 to 16 carbon atoms, and a substituent of each of R₅ and R₆ is a linear or branched alkyl group having 1 to 6 carbon atoms, a cyclohexyl group, a phenyl group, or a halogen, and
“I” represents an integer of from 1 to 5, and
wherein B represents (a) the substituted or unsubstituted alkylene group having 1 to 16 carbon atoms.
Item 5. The compound according to Item 1 or 2,
wherein A has the structure in which the two groups (c) are linked to each other, and
wherein B represents a group represented by the formula (2a):

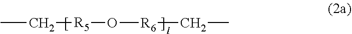

where R₅ and R₆ each independently represent a substituted or unsubstituted alkylene group having 1 to 16 carbon atoms, and a substituent of each of R₅ and R₆ is a linear or branched alkyl group having 1 to 6 carbon atoms, a cyclohexyl group, a phenyl group, or a halogen, and
“I” represents an integer of from 1 to 5.
Item 6. A polymer, which is obtained by polymerizing the compound of any one of Items 1 to 5.
Item 7. A polymer, which is represented by the following formula (11):

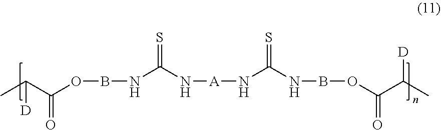

in the formula (11), A and B satisfy a relationship (I) or (II):
(I) one of A or B represents a group represented by the following formula (2):

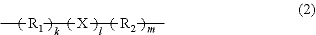

in the formula (2),
R₁ and R₂ each independently represent a substituted or unsubstituted alkylene group having 1 to 16 carbon atoms,
X is selected from the following groups:

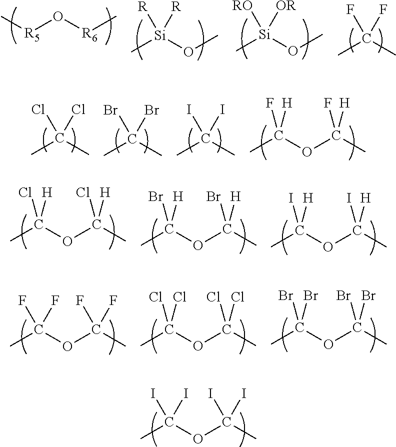

R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “I” represents an integer of 1 or more, and “m” represents an integer of 0 or more,
another of A or B represents one group selected from the following (a) to (c), or has a structure in which the following two or more groups (b) are linked to each other, or a structure in which the following two groups (c) are linked to each other:
(a) a substituted or unsubstituted alkylene group having 1 to 16 carbon atoms; (b):

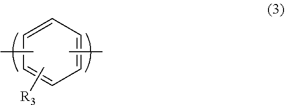

when one bonding moiety of main chain bonding moieties is defined as a 1-position, another bonding moiety thereof may be any one of a 2-position, a 3-position, and a 4-position, and R₃ represents a hydrogen atom, or 1 to 4 monovalent, identical or different, and substituted or unsubstituted alkyl groups each having 1 to 6 carbon atoms; and
(c):

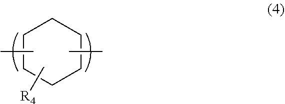

when one bonding moiety of main chain bonding moieties is defined as a 1-position, another bonding moiety thereof may be any one of a 2-position, a 3-position, and a 4-position, and R₄ represents a hydrogen atom, or 1 to 4 monovalent, identical or different, and substituted or unsubstituted alkyl groups each having 1 to 6 carbon atoms, and
when the other of A or B has the structure in which the following two or more groups (b) are linked to each other, or the structure in which the following two groups (c) are linked to each other, a linking group is —O— or —C(Rₐ)(R_{b})—, and Rₐ and R_{b} each independently represent hydrogen or an alkyl group having 1 to 6 carbon atoms; and
(II) both of A and B each independently represent a group represented by the following formula (2):

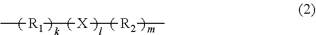

in the formula (2),
R₁ and R₂ each independently represent a substituted or unsubstituted alkylene group having 1 to 16 carbon atoms,
X is selected from the following groups:

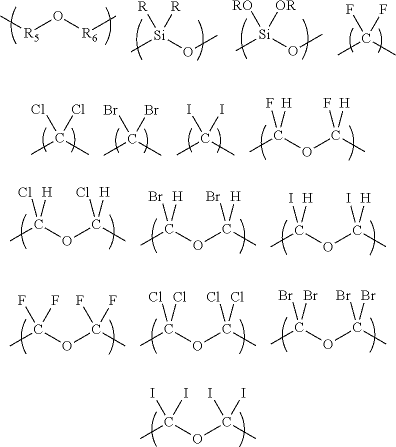

R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “I” represents an integer of 1 or more, and “m” represents an integer of 0 or more,
D represents hydrogen or a methyl group, and
“n” represents a polymerization degree of a repeating unit.
Item 8. An adhesive including the compound of any one of Items 1 to 5, or the polymer according to Item 6 or 7.
Item 9. An adhesive including:
the compound of any one of Items 1 to 5; and
a compound having two or more thiol groups.
Item 10. A bonding method including:
applying an adhesive including the compound of any one of Items 1 to 5 to a surface of a target; and
curing the applied adhesive by irradiating light to the adhesive.
Item 11. The bonding method according to Item 10,
wherein the target is a first target,
wherein the bonding method further includes bringing a second target into contact with the adhesive after the step of applying the adhesive so that the adhesive is arranged between the first target and the second target, and
wherein the applied adhesive is cured through the application of the light to the adhesive after the step of bringing the second target into contact with the adhesive.
Item 12. A bonding method comprising:
mixing the compound of any one of Items 1 to 5 and a compound having two or more thiol groups; and
applying the mixture to a surface of a target, followed by curing of the mixture.
Item 13. The bonding method according to Item 11 or 12, wherein the curing step is performed in water, in an aqueous solution, in an aqueous suspension, or in a high-humidity environment.
Item 14. The bonding method according to any one of Items 10 to 13, wherein the target is a material selected from the group consisting of: a metal, glass, wood and a synthetic resin.
Item 15. A use of the compound of any one of Items 1 to 5 for production of an adhesive.

### Advantageous Effects of Invention

The compound of the present invention may be used as an underwater adhesive, which can be rapidly cured in water and has a high underwater adhesive property.

### BRIEF DESCRIPTION OF DRAWINGS

FIG. 1 is an explanatory view of the roles of the respective moieties of a polymer of an embodiment of the present invention.

FIG. 2A is an explanatory view of a test for an underwater adhesive property between two different materials.

FIG. 2B is a graph of adhesive strengths to various materials.

FIG. 3 shows the adhesive strengths of samples bonded through three different processes.

FIG. 4A is an illustration of the structural formulae of monomers used in Test Example 3.

FIG. 4B is a graph of the underwater adhesive strengths of the respective monomers.

FIG. 5 is a graph of the underwater adhesive strengths of TUac-EG3 and T-Cy2 immediately after their bonding and 24 hours thereafter.

FIG. 6 shows a comparison between the water absorption behaviors of TUac-EG3 and T3EG.

FIG. 7 shows the dependence of the underwater adhesive property on the type of a cross-linker.

FIG. 8 shows the dependence of the underwater adhesive property on a curing time.

FIG. 9 shows the dependence of the underwater adhesive property on a monomer.

FIG. 10 shows a time-dependent underwater adhesion test.

FIG. 11 shows a time-dependent seawater adhesion test.

FIG. 12 shows the dependence of the underwater adhesive property on a substrate.

### DESCRIPTION OF EMBODIMENTS

In this description, a singular form (a, an, or the) includes a singular number and a plural number except for a case in which an explicit description is separately made in this description or a case in which a clear contradiction occurs in terms of context.

The term “comprise” as used herein is a concept comprehending “consist essentially of” and “consist of.”

In numerical ranges described herein in stages, the upper limit value or lower limit value of a numerical range at a certain stage may be arbitrarily combined with the upper limit value or lower limit value of a numerical range at any other stage. In addition, in a numerical range described herein, the upper limit value or lower limit value of the numerical range may be replaced with a value described in Examples or a value that may be uniquely derived from Examples. Further, in this description, numerical values tied to each other through “to” mean a numerical range including the numerical values in front of and behind “to” as a lower limit value and an upper limit value.

The term “(meth)acrylic acid” as used herein means acrylic acid and/or methacrylic acid.

Embodiments of the present invention are described in detail below. However, the present invention is by no means limited to the embodiments.

According to an aspect of the present invention, there is provided a compound, which is represented by the following formula (1):

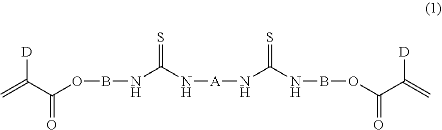

in the formula (1), A and B satisfy a relationship (1) or (II):
(1) one of A or B represents a group represented by the following formula (2):

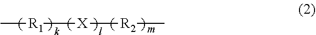

in the formula (2),
R₁ and R₂ each independently represent a substituted or unsubstituted alkylene group having 1 to 16 carbon atoms,
X is selected from the following groups:

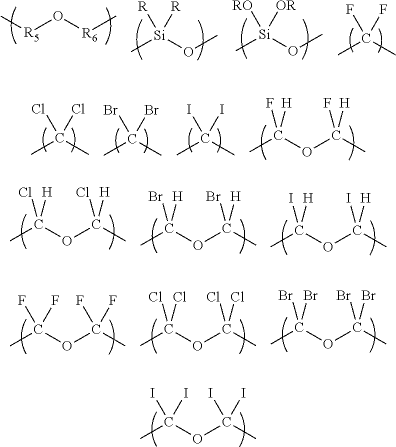

R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “I” represents an integer of 1 or more, and “m” represents an integer of 0 or more,
the other of A or B represents one group selected from the following (a) to (c), or has a structure in which the following two or more groups (b) are linked to each other, or a structure in which the following two groups (c) are linked to each other:
(a) a substituted or unsubstituted alkylene group having 1 to 16 carbon atoms; (b):

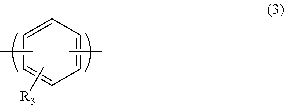

when one bonding moiety of main chain bonding moieties is defined as a 1-position, the other bonding moiety thereof may be any one of a 2-position, a 3-position, and a 4-position, and R₃ represents a hydrogen atom, or 1 to 4 monovalent, identical or different, and substituted or unsubstituted alkyl groups each having 1 to 6 carbon atoms; and
(c):

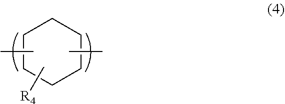

when one bonding moiety of main chain bonding moieties is defined as a 1-position, the other bonding moiety thereof may be any one of a 2-position, a 3-position, and a 4-position, and R₄ represents a hydrogen atom, or 1 to 4 monovalent, identical or different, and substituted or unsubstituted alkyl groups each having 1 to 6 carbon atoms, and
when the other of A or B has the structure in which the following two or more groups (b) are linked to each other, or the structure in which the following two groups (c) are linked to each other, a linking group is —O— or —C(Rₐ)(R_{b})—, and Rₐ and R_{b} each independently represent hydrogen or an alkyl group having 1 to 6 carbon atoms; and
(II) both of A and B each independently represent a group represented by the following formula (2):

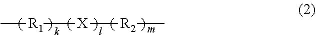

in the formula (2),
R₁ and R₂ each independently represent a substituted or unsubstituted alkylene group having 1 to 16 carbon atoms,
X is selected from the following groups:

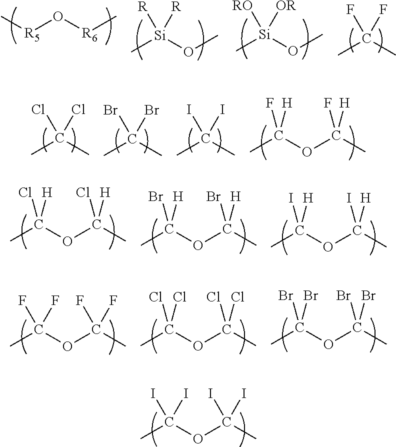

R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “I” represents an integer of 1 or more, and “m” represents an integer of 0 or more, and
D represents hydrogen or a methyl group.

In the formula (2), R₁ and R₂ each independently represent preferably a substituted or unsubstituted alkylene group having 2 to 12 carbon atoms, more preferably a substituted or unsubstituted alkylene group having 2 to 8 carbon atoms, still more preferably a substituted or unsubstituted alkylene group having 2 to 6 carbon atoms. It is still more preferred that R₁ and R₂ each independently represent a methylene group (—CH₂—) or an ethylene group (—CH₂—CH₂—), and it is still more preferred that both of R₁ and R₂ represent methylene groups (—CH₂—) or ethylene groups (—CH₂—CH₂—).

Examples of the substituents of R₁ and R₂ include a linear or branched alkyl group having 1 to 6 carbon atoms, a cyclohexyl group, a phenyl group, and a halogen (e.g., fluorine, chlorine, bromine, or iodine).

In the formula (2), “k” represents preferably from 0 to 3, more preferably from 0 to 2, still more preferably 0 or 1.

In the formula (2), “1” represents preferably from 1 to 5, more preferably from 1 to 4, still more preferably from 1 to 3.

In the formula (2), “m” represents preferably from 0 to 3, more preferably from 0 to 2, still more preferably 0 or 1.

In some embodiments, in the formula (2), “k” and “m” each represent 0 or 1, and “I” represents from 1 to 5.

In some embodiments, in the formula (2), “k” and “m” each represent 0 or 1, and “I” represents an integer of from 1 to 5, R₁ and R₂ each independently represent a substituted or unsubstituted alkylene group having 1 to 16 carbon atoms, X represents —R₅—O—R₆—, and R₅ and R₆ each independently represent a substituted or unsubstituted alkylene group having 1 to 16 carbon atoms.

In some embodiments, the group represented by the formula (2) is a group represented by the following formula (2a):

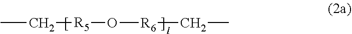

where R₅ and R₆ each independently represent a substituted or unsubstituted alkylene group having 1 to 16 carbon atoms, and “I” represents an integer of from 1 to 5.
R₅ and R₆ each independently represent preferably a substituted or unsubstituted alkylene group having 2 to 12 carbon atoms, more preferably a substituted or unsubstituted alkylene group having 2 to 8 carbon atoms, still more preferably a substituted or unsubstituted alkylene group having 2 to 6 carbon atoms.

Examples of the substituents of R₅ and R₆ include a linear or branched alkyl group having 1 to 6 carbon atoms, a cyclohexyl group, a phenyl group, and a halogen (e.g., fluorine, chlorine, bromine, or iodine).

In some embodiments, in the formula (2), “k” and “m” each represent 0 or 1, and “I” represents an integer of from 1 to 5, R₁ and R₂ each independently represent a substituted or unsubstituted alkylene group having 1 to 16 carbon atoms, X represents —R₆—O—R₆—, and R₅ and R₆ each independently represent a divalent group represented by —CₚH₂ₚ— (where “p” represents an integer of from 1 to 3).

In a preferred embodiment, in the compound represented by the formula (1), A and B satisfy a relationship (I) or (II):
(I) one of A or B represents a group represented by the following formula (2a):

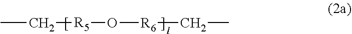

where R₅ and R₆ each independently represent a substituted or unsubstituted alkylene group having 1 to 16 carbon atoms, and “I” represents an integer of from 1 to 5,
the other of A or B represents one group selected from the following (a) to (c), or has a structure in which the following two or more groups (b) are linked to each other, or a structure in which the following two groups (c) are linked to each other:
(a) a substituted or unsubstituted alkylene group having 1 to 16 carbon atoms;
(b):

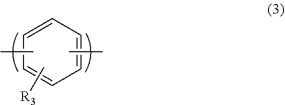

when one bonding moiety of main chain bonding moieties is defined as a 1-position, the other bonding moiety thereof may be any one of a 2-position, a 3-position, and a 4-position, and R₃ represents a hydrogen atom, or 1 to 4 monovalent, identical or different, and substituted or unsubstituted alkyl groups each having 1 to 6 carbon atoms; and
(c):

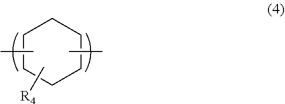

when one bonding moiety of main chain bonding moieties is defined as a 1-position, the other bonding moiety thereof may be any one of a 2-position, a 3-position, and a 4-position, and R₄ represents a hydrogen atom, or 1 to 4 monovalent, identical or different, and substituted or unsubstituted alkyl groups each having 1 to 6 carbon atoms, and
when the other of A or B has the structure in which the following two or more groups (b) are linked to each other, or the structure in which the following two groups (c) are linked to each other, a linking group is —O— or —C(Rₐ)(R_{b})—, and Rₐ and R_{b} each independently represent hydrogen or an alkyl group having 1 to 6 carbon atoms; and
(II) both of A and B each independently represent a group represented by the following formula (2a):

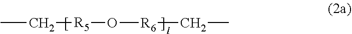

where R₅ and R₆ each independently represent a substituted or unsubstituted alkylene group having 1 to 16 carbon atoms, and “I” represents an integer of from 1 to 5, and
D represents hydrogen or a methyl group.

In the formula (2a), R₅ and R₆ each independently represent preferably a substituted or unsubstituted alkylene group having 1 to 12 carbon atoms, more preferably a substituted or unsubstituted alkylene group having 1 to 8 carbon atoms, still more preferably a substituted or unsubstituted alkylene group having 1 to 6 carbon atoms, still more preferably a substituted or unsubstituted alkylene group having 1 to 3 carbon atoms.

Examples of the substituents of R₅ and R₆ include a linear or branched alkyl group having 1 to 6 carbon atoms, a cyclohexyl group, a phenyl group, and a halogen (e.g., fluorine, chlorine, bromine, or iodine).

(a) The substituted or unsubstituted alkylene group having 1 to 16 carbon atoms is preferably a substituted or unsubstituted alkylene group having 2 to 12 carbon atoms, more preferably a substituted or unsubstituted alkylene group having 2 to 8 carbon atoms, still more preferably a substituted or unsubstituted alkylene group having 2 to 6 carbon atoms.

A substituent of (a) the substituted alkylene group having 1 to 16 carbon atoms is, for example, a linear or branched alkyl group having 1 to 6 carbon atoms, a cyclohexyl group, a phenyl group, or a halogen (e.g., fluorine, chlorine, bromine, or iodine). In addition, the number of the substituents in (a) the substituted or unsubstituted alkylene group having 1 to 16 carbon atoms is preferably from 1 to 3.

When R₃ in (b) the group represented by the formula (3) represents a substituted alkyl group having 1 to 6 carbon atoms, a substituent thereof is, for example, a linear or branched alkyl group having 1 to 6 carbon atoms, or a halogen (e.g., fluorine, chlorine, bromine, or iodine).

In some embodiments, (b) the group represented by the formula (3) is a group represented by the following formula (3a):

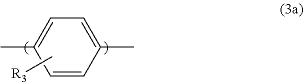

where R₃ represents a hydrogen atom, or 1 to 4 monovalent and identical alkyl groups each having 1 to 6 carbon atoms.

When R₄ in (c) the group represented by the formula (4) represents a substituted alkyl group having 1 to 6 carbon atoms, a substituent thereof is, for example, a linear or branched alkyl group having 1 to 6 carbon atoms, or a halogen (e.g., fluorine, chlorine, bromine, or iodine).

In some embodiments, (c) the group represented by the formula (4) is a group represented by the following formula (4a):

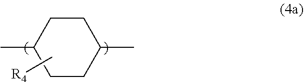

where R₄ represents a hydrogen atom, or 1 to 4 monovalent alkyl groups each having 1 to 6 carbon atoms. In some embodiments, when the other of A or B represents a structure in which the two or more groups (b) are linked to each other, (b) the group represented by the formula (3) is a group represented by the following formula (3a), a linking group is —O— or —C(Rₐ)(R_{b})—, and Rₐ and R_{b} each independently represent hydrogen or an alkyl group having 1 to 6 carbon atoms.

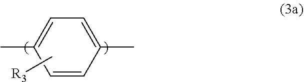

where R₃ represents a hydrogen atom, or 1 to 4 monovalent and identical alkyl groups each having 1 to 6 carbon atoms.

In some embodiments, when the other of A or B represents a structure in which the two or more groups (b) are linked to each other, the structure in which the two or more groups (b) are linked to each other is a structure represented by the following formula (5) or (6).

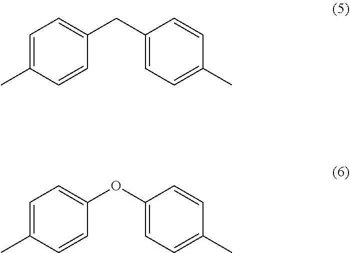

In some embodiments, when the other of A or B represents a structure in which the two or more groups (c) are linked to each other, (c) the group represented by the formula (4) is a group represented by the following formula (4a), a linking group is —O— or —C(Rₐ)(R_{b})—, and Rₐ and R_{b} each independently represent hydrogen or an alkyl group having 1 to 6 carbon atoms:

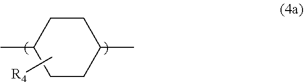

where R₄ represents a hydrogen atom, or 1 to 4 monovalent and identical alkyl groups each having 1 to 6 carbon atoms.

In some embodiments, when the other of A or B represents a structure in which the two or more groups (c) are linked to each other, the structure in which the two or more groups (c) are linked to each other is a structure represented by the following formula (7) or (8).

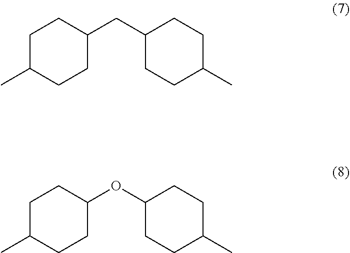

D may represent hydrogen or a methyl group. In some embodiments, D represents hydrogen. In some embodiments, D represents a methyl group.

In some embodiments, the compound represented by the formula (1) is a compound obtained by polymerizing a compound represented by the following formula (9) and a compound represented by the following formula (10).

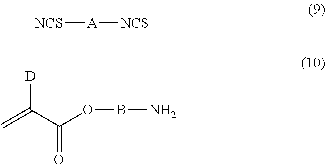

A in the formula (9) and B in the formula (10) satisfy a relationship (I) or (II): (I) one of A or B represents a group represented by the following formula (2):

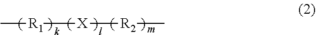

in the formula (2),
R₁ and R₂ each independently represent a substituted or unsubstituted alkylene group having 1 to 16 carbon atoms,
X is selected from the following groups:

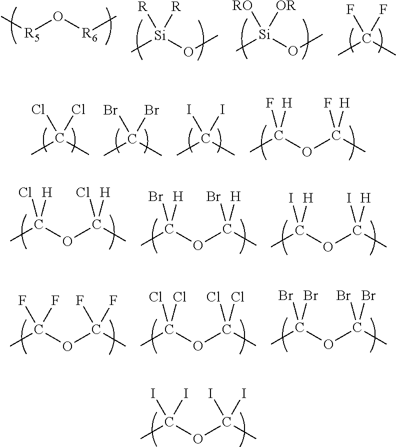

R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “I” represents an integer of 1 or more, and “m” represents an integer of 0 or more,
the other of A or B represents one group selected from the following (a) to (c), or has a structure in which the following two or more groups (b) are linked to each other, or a structure in which the following two groups (c) are linked to each other:
(a) a substituted or unsubstituted alkylene group having 1 to 16 carbon atoms; (b):

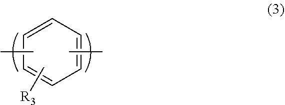

when one bonding moiety of main chain bonding moieties is defined as a 1-position, the other bonding moiety thereof may be any one of a 2-position, a 3-position, and a 4-position, and R₃ represents a hydrogen atom, or 1 to 4 monovalent, identical or different, and substituted or unsubstituted alkyl groups each having 1 to 6 carbon atoms; and
(c):

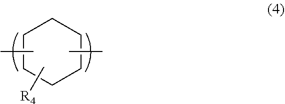

when one bonding moiety of main chain bonding moieties is defined as a 1-position, the other bonding moiety thereof may be any one of a 2-position, a 3-position, and a 4-position, and R₄ represents a hydrogen atom, or 1 to 4 monovalent, identical or different, and substituted or unsubstituted alkyl groups each having 1 to 6 carbon atoms, and
when the other of A or B has the structure in which the following two or more groups (b) are linked to each other, or the structure in which the following two groups (c) are linked to each other, a linking group is —O— or —C(Rₐ)(R_{b})—, and Rₐ and R_{b} each independently represent hydrogen or an alkyl group having 1 to 6 carbon atoms; and
(II) both of A and B each independently represent a group represented by the following formula (2):

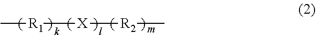

in the formula (2),
R₁ and R₂ each independently represent a substituted or unsubstituted alkylene group having 1 to 16 carbon atoms,
X is selected from the following groups:

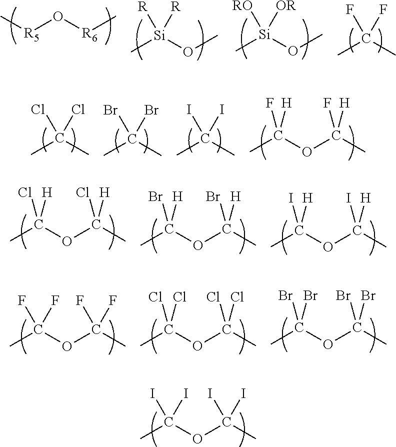

R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “I” represents an integer of 1 or more, and “m” represents an integer of 0 or more, and
D represents hydrogen or a methyl group.

In the formula (2), R₁ and R₂ each independently represent preferably a substituted or unsubstituted alkylene group having 2 to 12 carbon atoms, more preferably a substituted or unsubstituted alkylene group having 2 to 8 carbon atoms, still more preferably a substituted or unsubstituted alkylene group having 2 to 6 carbon atoms. It is still more preferred that R₁ and R₂ each independently represent a methylene group (—CH₂—) or an ethylene group (—CH₂—CH₂—), and it is still more preferred that both of R₁ and R₂ represent methylene groups (—CH₂—) or ethylene groups (—CH₂—CH₂—).

Examples of the substituents of R₁ and R₂ include a linear or branched alkyl group having 1 to 6 carbon atoms, a cyclohexyl group, a phenyl group, and a halogen (e.g., fluorine, chlorine, bromine, or iodine).

In the formula (2), “k” represents preferably from 0 to 3, more preferably from 0 to 2, still more preferably 0 or 1.

In the formula (2), “I” represents preferably from 1 to 5, more preferably from 1 to 4, still more preferably from 1 to 3.

In the formula (2), “m” represents preferably from 0 to 3, more preferably from 0 to 2, still more preferably 0 or 1.

In some embodiments, in the formula (2), “k” and “m” each represent 0 or 1, and “I” represents from 1 to 5.

In some embodiments, in the formula (2), “k” and “m” each represent 0 or 1, and “I” represents an integer of from 1 to 5, R₁ and R₂ each independently represent a substituted or unsubstituted alkylene group having 1 to 16 carbon atoms, X represents —R₆—O—R₆—, and R₅ and R₆ each independently represent a substituted or unsubstituted alkylene group having 1 to 16 carbon atoms.

In some embodiments, the group represented by the formula (2) is a group represented by the following formula (2a):

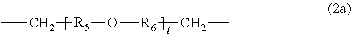

where R₅ and R₆ each independently represent a substituted or unsubstituted alkylene group having 1 to 16 carbon atoms, and “I” represents an integer of from 1 to 5.
R₅ and R₆ each independently represent preferably a substituted or unsubstituted alkylene group having 2 to 12 carbon atoms, more preferably a substituted or unsubstituted alkylene group having 2 to 8 carbon atoms, still more preferably a substituted or unsubstituted alkylene group having 2 to 6 carbon atoms.

Examples of the substituents of R₅ and R₆ include a linear or branched alkyl group having 1 to 6 carbon atoms, a cyclohexyl group, a phenyl group, and a halogen (e.g., fluorine, chlorine, bromine, or iodine).

In some embodiments, in the formula (2), “k” and “m” each represent 0 or 1, and “I” represents an integer of from 1 to 5, R₁ and R₂ each independently represent a substituted or unsubstituted alkylene group having 1 to 16 carbon atoms, X represents —R₆—O—R₆—, and R₅ and R₆ each independently represent a divalent group represented by —CₚH₂ₚ— (where “p” represents an integer of from 1 to 3).

In a preferred embodiment, A in the formula (9) and B in the formula (10) satisfy a relationship (I) or (II):
(I) one of A or B represents a group represented by the following formula (2a):

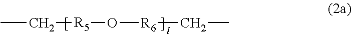

where R₅ and R₆ each independently represent a divalent group represented by —CₚH₂ₚ— (where “p” represents an integer of from 1 to 3), and “I” represents an integer of from 1 to 5,
the other of A or B represents one group selected from the following (a) to (c), or has a structure in which the following two or more groups (b) are linked to each other, or a structure in which the following two groups (c) are linked to each other:
(a) a substituted or unsubstituted alkylene group having 1 to 16 carbon atoms; (b):

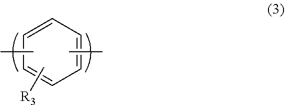

when one bonding moiety of main chain bonding moieties is defined as a 1-position, the other bonding moiety thereof may be any one of a 2-position, a 3-position, and a 4-position, and R₃ represents a hydrogen atom, or 1 to 4 monovalent, identical or different, and substituted or unsubstituted alkyl groups each having 1 to 6 carbon atoms; and
(c):

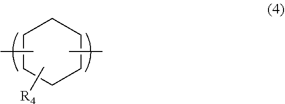

when one bonding moiety of main chain bonding moieties is defined as a 1-position, the other bonding moiety thereof may be any one of a 2-position, a 3-position, and a 4-position, and R₄ represents a hydrogen atom, or 1 to 4 monovalent, identical or different, and substituted or unsubstituted alkyl groups each having 1 to 6 carbon atoms, and
when the other of A or B has the structure in which the following two or more groups (b) are linked to each other, or the structure in which the following two groups (c) are linked to each other, a linking group is —O— or —C(Rₐ)(R_{b})—, and Rₐ and R_{b} each independently represent hydrogen or an alkyl group having 1 to 6 carbon atoms; and
(II) both of A and B each independently represent a group represented by the following formula (2a):

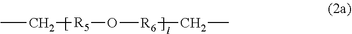

where R₅ and R₆ each independently represent a divalent group represented by —CₚH₂ₚ— (where “p” represents an integer of from 1 to 3), and “I” represents an integer of from 1 to 5.
(a) The substituted or unsubstituted alkylene group having 1 to 16 carbon atoms is preferably a substituted or unsubstituted alkylene group having 2 to 12 carbon atoms, more preferably a substituted or unsubstituted alkylene group having 2 to 8 carbon atoms, still more preferably a substituted or unsubstituted alkylene group having 2 to 6 carbon atoms.

A substituent of (a) the substituted alkylene group having 1 to 16 carbon atoms is, for example, a linear or branched alkyl group having 1 to 6 carbon atoms, a cyclohexyl group, a phenyl group, or a halogen (e.g., fluorine, chlorine, bromine, or iodine). In addition, the number of the substituents in (a) the substituted or unsubstituted alkylene group having 1 to 16 carbon atoms is preferably from 1 to 3.

When R₃ in (b) the group represented by the formula (3) represents a substituted alkyl group having 1 to 6 carbon atoms, a substituent thereof is, for example, a linear or branched alkyl group having 1 to 6 carbon atoms, or a halogen (e.g., fluorine, chlorine, bromine, or iodine).

In some embodiments, (b) the group represented by the formula (3) is a group represented by the following formula (3a):

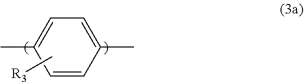

where R₃ represents a hydrogen atom, or 1 to 4 monovalent and identical alkyl groups each having 1 to 6 carbon atoms.

When R₄ in (c) the group represented by the formula (4) represents a substituted alkyl group having 1 to 6 carbon atoms, a substituent thereof is, for example, a linear or branched alkyl group having 1 to 6 carbon atoms, or a halogen (e.g., fluorine, chlorine, bromine, or iodine).

In some embodiments, (c) the group represented by the formula (4) is a group represented by the following formula (4a):

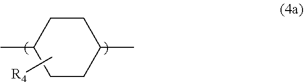

where R₄ represents a hydrogen atom, or 1 to 4 monovalent alkyl groups each having 1 to 6 carbon atoms.

The compound obtained by polymerizing the compound represented by the formula (9) and the compound represented by the formula (10) may contain, as a monomer component, a compound except the compound represented by the formula (9) and the compound represented by the formula (10), or may be free of the compound.

In some embodiments, the compound obtained by polymerizing the compound represented by the formula (9) and the compound represented by the formula (10) contains, as monomer components, only the compound represented by the formula (9) and the compound represented by the formula (10).

In some embodiments, when the other of A or B represents a structure in which the two or more groups (b) are linked to each other, (b) the group represented by the formula (3) is a group represented by the following formula (3a), a linking group is —O— or —C(Rₐ)(R_{b})—, and Rₐ and R_{b} each independently represent hydrogen or an alkyl group having 1 to 6 carbon atoms:

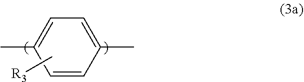

where R₃ represents a hydrogen atom, or 1 to 4 monovalent and identical alkyl groups each having 1 to 6 carbon atoms.

In some embodiments, when the other of A or B represents a structure in which the two or more groups (b) are linked to each other, the structure in which the two or more groups (b) are linked to each other is a structure represented by the following formula (5) or (6).

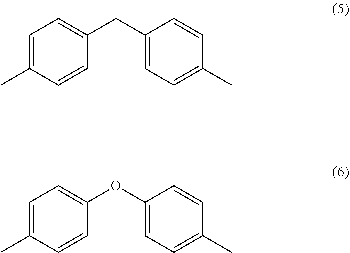

In some embodiments, when the other of A or B represents a structure in which the two or more groups (c) are linked to each other, (c) the group represented by the formula (4) is a group represented by the following formula (4a), a linking group is —O— or—C(Rₐ)(R_{b})—, and Rₐ and R_{b} each independently represent hydrogen or an alkyl group having 1 to 6 carbon atoms:

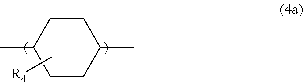

where R₄ represents a hydrogen atom, or 1 to 4 monovalent and identical alkyl groups each having 1 to 6 carbon atoms.

In some embodiments, when the other of A or B represents a structure in which the two or more groups (c) are linked to each other, the structure in which the two or more groups (c) are linked to each other is a structure represented by the following formula (7) or (8).

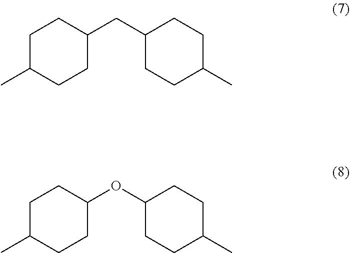

D may represent hydrogen or a methyl group. In some embodiments, D represents hydrogen. In some embodiments, D represents a methyl group.

In some embodiments of the compound represented by the formula (1), A represents a group represented by the formula (2), “k” and “m” each represent 0 or 1, and “I” represents an integer of from 1 to 5, R₁ and R₂ each independently represent a substituted or unsubstituted alkylene group having 1 to 16 carbon atoms, X represents —R₆—O—R₆—, R₅ and R₆ each independently represent a substituted or unsubstituted alkylene group having 1 to 16 carbon atoms, and B represents (a) the substituted or unsubstituted alkylene group having 1 to 16 carbon atoms.

In some embodiments of the compound represented by the formula (1), A represents a group represented by the formula (2a), R₅ and R₆ each independently represent a divalent group represented by —CₚH₂ₚ— (where “p” represents an integer of from 1 to 3), “1” represents an integer of from 1 to 5, and B represents (a) the substituted or unsubstituted alkylene group having 1 to 16 carbon atoms.

In the formula (2a), it is preferred that R₁ and R₂ each independently represent a methylene group (—CH₂—) or an ethylene group (—CH₂—CH₂—), and it is more preferred that both of R₁ and R₂ represent methylene groups (—CH₂—) or ethylene groups (—CH₂—CH₂—).

In the formula (2a), “I” represents preferably from 1 to 4, more preferably from 1 to 3.
(a) The substituted or unsubstituted alkylene group having 1 to 16 carbon atoms is preferably a substituted or unsubstituted alkylene group having 2 to 12 carbon atoms, more preferably a substituted or unsubstituted alkylene group having 2 to 8 carbon atoms, still more preferably a substituted or unsubstituted alkylene group having 2 to 6 carbon atoms, still more preferably an alkylene group having 2 to 6 carbon atoms.

A substituent of (a) the substituted alkylene group having 1 to 16 carbon atoms is, for example, a linear or branched alkyl group having 1 to 6 carbon atoms, a cyclohexyl group, a phenyl group, or a halogen (e.g., fluorine, chlorine, bromine, or iodine). In addition, the number of the substituents in (a) the substituted or unsubstituted alkylene group having 1 to 16 carbon atoms is preferably from 1 to 3.

In a specific embodiment, in the formula (2a), R₁ and R₂ each represent a methylene group, “I” represents from 1 to 3, and the group (a) is an alkylene group having 2 to 6 carbon atoms.

In a specific embodiment, in the formula (2a), R₁ and R₂ each represent a methylene group, “I” represents 1 or 2, and the group (a) is an alkylene group having 2 carbon atoms.

In some embodiments of the compound represented by the formula (1), A has a structure in which the two groups (c) are linked to each other, and B represents a group represented by the formula (2):

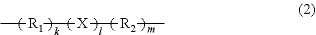

where
R₁ and R₂ each independently represent a substituted or unsubstituted alkylene group having 1 to 16 carbon atoms,
X is selected from the following groups:

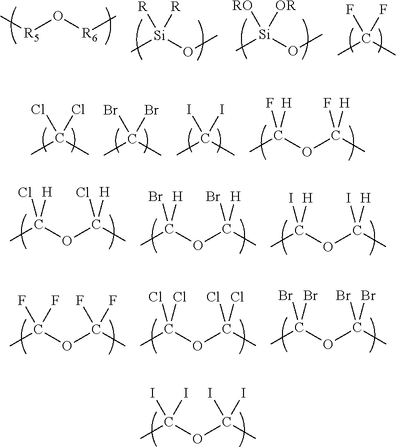

R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “I” represents an integer of 1 or more, and “m” represents an integer of 0 or more.

The structure in which the two or more groups (c) are linked to each other is preferably a structure represented by the following formula (7) or (8).

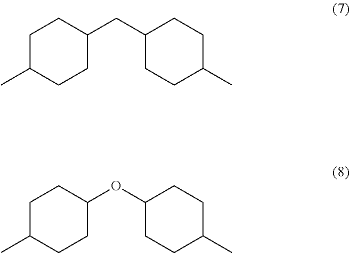

In a specific embodiment, the structure represented by A in which the two or more groups (c) are linked to each other is a structure represented by the formula (7) or (8), B represents a group represented by the formula (2a), R₅ and R₆ each represent a methylene group or an ethylene group, and “I” represents from 1 to 3.

In a specific embodiment, the structure represented by A in which the two or more groups (c) are linked to each other is a structure represented by the formula (7), B represents a group represented by the formula (2a), R₅ and R₆ each represent a methylene group, and “I” represents 1 or 2.

According to another aspect of the present invention, there is provided a polymer, which is obtained by polymerizing the compound represented by the formula (1).

According to another aspect of the present invention, there is provided a polymer, which is represented by the following formula (11):

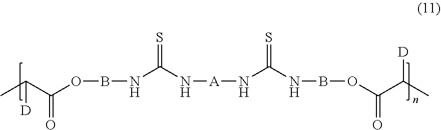

in the formula (11), A and B satisfy a relationship (I) or (II):
(I) one of A or B represents a group represented by the following formula (2):

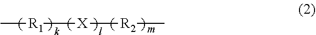

in the formula (2),
R₁ and R₂ each independently represent a substituted or unsubstituted alkylene group having 1 to 16 carbon atoms,
X is selected from the following groups:

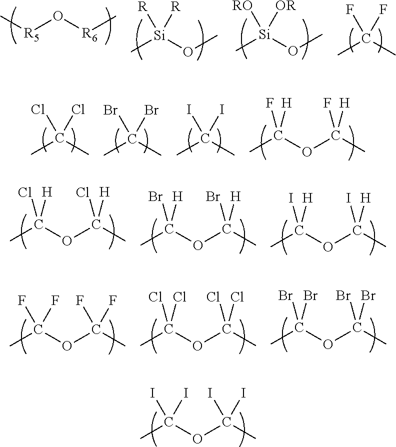

R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “I” represents an integer of 1 or more, and “m” represents an integer of 0 or more,
the other of A or B represents one group selected from the following (a) to (c), or has a structure in which the following two or more groups (b) are linked to each other, or a structure in which the following two groups (c) are linked to each other:
(a) a substituted or unsubstituted alkylene group having 1 to 16 carbon atoms; (b):

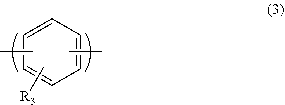

when one bonding moiety of main chain bonding moieties is defined as a 1-position, the other bonding moiety thereof may be any one of a 2-position, a 3-position, and a 4-position, and R₃ represents a hydrogen atom, or 1 to 4 monovalent, identical or different, and substituted or unsubstituted alkyl groups each having 1 to 6 carbon atoms; and
(c):

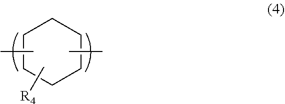

when one bonding moiety of main chain bonding moieties is defined as a 1-position, the other bonding moiety thereof may be any one of a 2-position, a 3-position, and a 4-position, and R₄ represents a hydrogen atom, or 1 to 4 monovalent, identical or different, and substituted or unsubstituted alkyl groups each having 1 to 6 carbon atoms, and
when the other of A or B has the structure in which the following two or more groups (b) are linked to each other, or the structure in which the following two groups (c) are linked to each other, a linking group is —O— or —C(Rₐ)(R_{b})—, and Rₐ and R_{b} each independently represent hydrogen or an alkyl group having 1 to 6 carbon atoms; and
(II) both of A and B each independently represent a group represented by the following formula (2):

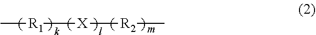

in the formula (2),
R₁ and R₂ each independently represent a substituted or unsubstituted alkylene group having 1 to 16 carbon atoms,
X is selected from the following groups:

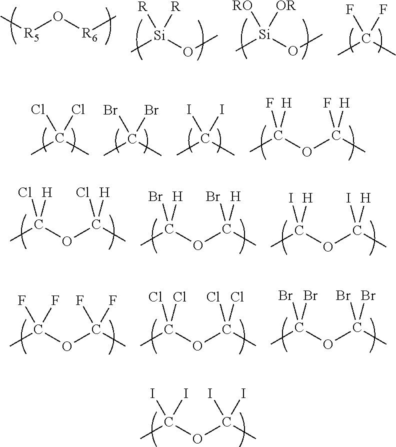

R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “I” represents an integer of 1 or more, and “m” represents an integer of 0 or more.
D represents hydrogen or a methyl group.
“n” represents a polymerization degree of a repeating unit.

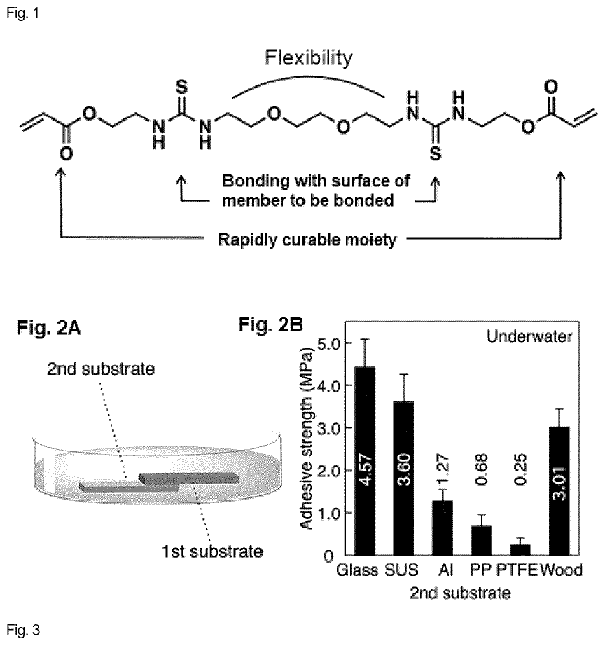

Although the applicant of the present application does not wish the present invention to be bound by a specific hypothesis or theory, as illustrated in FIG. 1, when the compound represented by the formula (1) is polymerized, it is conceivable that an ether imparts flexibility to the entire molecule, a thiourea structural unit has an ability to be bonded to the surface of a target to be bonded such as a glass substrate, and a structural unit derived from (meth)acrylic acid contributes to the rapid curing of the polymer. A polymer and an adhesive satisfying constituent features specified in the present invention are included in the technical scope of the present invention even when the polymer and the adhesive do not completely follow such theory.

Accordingly, the polymer obtained by polymerizing the compound represented by the formula (1) may be used as an adhesive. The adhesive may consist of the polymer obtained by polymerizing the compound represented by the formula (1), or may include an additive other than the polymer to the extent that characteristics as an adhesive are not impaired. When the adhesive of the embodiment of the present invention includes the additive other than the polymer obtained by polymerizing the compound represented by the formula (1), such adhesive may be referred to as an “adhesive composition.” An object of the invention is to provide a compound, which can be rapidly cured in water and has a high underwater adhesive property, and a polymer thereof.

In some embodiments, photoirradiation can quicken the polymerization and curing of the compound represented by the formula (1), and hence the adhesive including such compound may be used as a one-component adhesive.

In some embodiments, the compound represented by the formula (1) and a compound having two or more thiol groups may be used as a two-component adhesive. The two or more thiol groups each act as a cross-linker for the compound represented by the formula (1).

(Meth)acrylate groups at both the terminals of the compound represented by the formula (1) (R₁ represents such a hydrocarbon group that part of the carbon atoms of its main chain may each be substituted) are condensed with the thiol groups of the compound having two or more thiol groups (R₂ represents such a hydrocarbon group that part of the carbon atoms of its main chain may each be substituted) by a thiol-ene reaction to form a polymer. The thiol-ene reaction is known as a polymerization reaction between an alkene and a thiol. As represented by the following scheme A, for example, when an alkene represented by the formula (12) and a compound having two thiol groups represented by the formula (13) are caused to react with each other in the presence of a catalyst, the alkene and the compound are polymerized to form a three-dimensional network represented by the formula (14), and are cured. Such catalyst is known, and an arbitrary base or nucleophilic catalyst that accelerates the thiol-ene reaction may be used. A preferred example thereof is an amine catalyst. In the thiol-ene reaction, the carbon-carbon double bond of the alkene represented by the formula (12) and a thiol group of the compound represented by the formula (13) react with each other at 1:1. Accordingly, in the case where an adhesive is produced by the condensation between the compound represented by the formula (1) and the compound having two or more thiol groups, the adhesive can be produced even when an equivalent ratio (C═C:SH) between the carbon-carbon double bond of the alkene represented by the formula (12) and the thiol group of the compound represented by the formula (13) falls within the range of from 1:10 to 10:1. However, the ratio is more preferably from 1:3 to 3:1, most preferably 1:1.

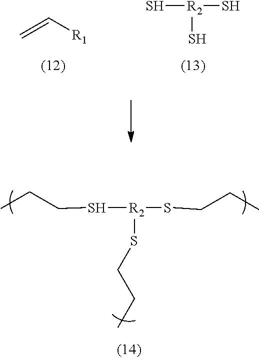

In some embodiments, the compound represented by the formula (1) may be polymerized by using a polymerization initiator and used as an adhesive. A known polymerization initiator that may be used in the polymerization of a (meth)acrylic ester monomer may be used as such polymerization initiator.

Examples of such polymerization initiator include a peroxide-based polymerization initiator, an azo polymerization initiator, a photopolymerization initiator, and combinations thereof.

Examples of the type of the peroxide-based polymerization initiator include, but not limited to, benzoyl peroxide, 1,1-bis-t-hexylperoxy-3,3,5-trimethylcyclohexane, and 3,5,5-trimethylhexanoyl peroxide.

Examples of the azo-based polymerization initiator include, but not limited to, 2,2′-azobisisobutyronitrile, 1,1′-azobiscydohexane-1-carbonitrile, and 2,2′-azobis-2,4-dimethylvaleronitrile.

The photopolymerization initiator is a polymerization initiator that initiates polymerization when irradiated with light, such as an electron beam, UV light, visible light, or near-infrared light. Such photopolymerization initiator is known in the art. The photopolymerization initiator may be a self-cleaving photopolymerization initiator, or may be a hydrogen abstraction-type photopolymerization initiator.

Examples of the self-cleaving photopolymerization initiator include, but not limited to, an alkylphenone-based compound, an acylphosphine oxide-based compound, a titanocene-based compound, an acetophenone-based compound, a phenylglyoxylate-based compound, and a benzoin ether-based compound.

Examples of the hydrogen abstraction-type photopolymerization initiator include, but not limited to, an oxime ester-based compound, a benzophenone-based compound, a thioxanthone-based compound, an anthraquinone-based compound, and a benzil-based compound.

The adhesive of the embodiment of the present invention may be used in the bonding of target made of a wide variety of materials. Even a material that is considered to be difficult to bond can be suitably bonded. A material for a target may be, for example, a metal, glass, wood, or a resin. The metal includes metal compounds, such as an alloy and a metal oxide. The resin includes a natural resin and a synthetic resin.

The adhesive of the embodiment of the present invention can not only be used in bonding in the air but also be used in bonding in water. The phrase “in water” includes the inside of the water, the inside of an aqueous solution, and the inside of an aqueous suspension. The term “bonding in water” as used herein includes the bonding of the surfaces of two targets, which are each in a state in which the water has adhered to its surface owing to a high-humidity environment, without through any drying process. That is, the term includes the bonding of the surfaces of targets wetted with the water.

Although the adhesive of the embodiment of the present invention can be naturally cured, photoirradiation can quicken its curing, and hence the adhesive is rapidly and easily cured in water. In addition, the adhesive of the embodiment of the present invention can be used in bonding without being melted by heating, and hence does not require a complicated process in which the adhesive alone is partially heated in the water for hot melting. In addition, the adhesive does not require any solvent, and hence its environmental load is small.

Further, after the bonding, the adhesive of the embodiment of the present invention can not only maintain the bonding in the air but also maintain the bonding in water. The polymer of the embodiment of the present invention has a moisture-absorbing property lower than that of a polymer free of a structural unit derived from (meth)acrylic acid. Accordingly, the adhesive can maintain a high underwater adhesive strength even when placed in the water for a long time period. For example, the adhesive of the embodiment of the present invention can maintain an adhesive strength in the water 24 hours after its application to the surface of a target at 80% or more of an adhesive strength in the water immediately after the application to the surface of the target. To the best of the inventors' knowledge, the underwater adhesive strength of the adhesive of the embodiment of the present invention is much larger than that of a commercial adhesive in terms of both of an underwater adhesive strength immediately after the application to the surface of the target and an underwater adhesive property after the lapse of a certain time period from the application to the surface of the target.

Further, after the bonding, the adhesive of the embodiment of the present invention can maintain the bonding even in seawater. For example, the adhesive of the embodiment of the present invention can maintain an adhesive strength in the seawater 24 hours after its application to the surface of a target at 80% or more of an adhesive strength in the seawater immediately after the application to the surface of the target.

The adhesive of the embodiment of the present invention may be suitably used as a repair agent, or a coating or lining agent through exploitation of its rapid curability, adhesive property, and stability under contact with water. In particular, the adhesive of the embodiment of the present invention may be used as a so-called all-underwater adhesive having a high underwater adhesive property even when a process from its application to its curing is performed in the water. Accordingly, the adhesive is applicable to various fields where all-underwater bonding is required including: the repair of an apparatus, such as a ship, a submarine, a power generator, or a submarine cable; and the repair of the water tank of a pool.

The adhesive of the embodiment of the present invention may be used in the bonding of a target.

In some embodiments, a bonding method including using the adhesive of the embodiment of the present invention includes applying the adhesive to the bonding surface of a target; and curing the applied adhesive by irradiating light to the adhesive. The light to be applied is preferably visible light, and the wavelength of the visible light is preferably from 360 nm to 830 nm, more preferably from 360 nm to 760 nm.

The bonding method may further include a step of fixing the applied adhesive after the applying step and before the curing step. The step of fixing the adhesive by bringing the adhesive into contact with the surface of the target to be bonded may be performed by known means. For example, the fixation may be performed by preparing a dedicated fixing tool.

In a specific embodiment, the target is formed of a first target and a second target, and the bonding method may include applying the adhesive to the bonding surface of the first targe; bringing the second target into contact with the adhesive after the step of applying the adhesive so that the adhesive is arranged between the first target and the second target; and curing the applied adhesive by irradiating light to the adhesive after the step of bringing the second target into contact with the adhesive. When the bonding method further includes a step of fixing the adhesive, the fixation may be performed by: performing such pressing that one of the first target or the second target between which the adhesive is arranged is pushed against the other; or sandwiching the first target and the second target between fixing tools, followed by pressing from both sides.

In some embodiments, the bonding method includes mixing the above-mentioned compound represented by the formula (1) with a compound having two or more thiol groups; and applying the mixture to the surface of the target, followed by the curing of the mixture.

The bonding method may further include fixing the applied adhesive after the applying step. The step of fixing the adhesive by bringing the adhesive into contact with the surface of the target may be performed by known means. For example, the fixation may be performed by preparing a dedicated fixing tool.

The applying step, the fixing step, and the curing step may each independently be performed in the air or in water. In a preferred embodiment, the applying step, the fixing step, and the curing step may be performed in the water. The bonding method of the embodiment of the present invention, which commences on the application of the adhesive to the bonding surface of the target and ends on its curing, can be completely performed in the water.

The adhesive of the embodiment of the present invention may be used in bonding by being polymerized and cured through heating. When the adhesive is applied to the bonding surface of the target by being heated and melted in the air, the adhesive may be applied to the bonding surface by being heated and melted.

The polymer of the present invention includes the repeating unit to exhibit the above-mentioned excellent characteristics. Accordingly, even when any other repeating unit is introduced or a side chain of the polymer is modified through use of known means to the extent that those excellent characteristics are not impaired, the resultant polymer falls within the scope of the present invention. In addition, the polymer of the present invention exhibits excellent characteristics as an adhesive even when used alone. Accordingly, even when a desired additive is added to the polymer for characteristic control through use of known means to the extent that those excellent characteristics are not impaired, a composition having added thereto the additive falls within the scope of the present invention.

### EXAMPLES

The present invention is more specifically described below by way of Examples. However, the present invention is not limited thereto.

### Production Example 1: Synthesis of TUac-EG3

TUac-EG3 was synthesized as a monomer of Production Example 1 through a route represented by the following scheme 1.

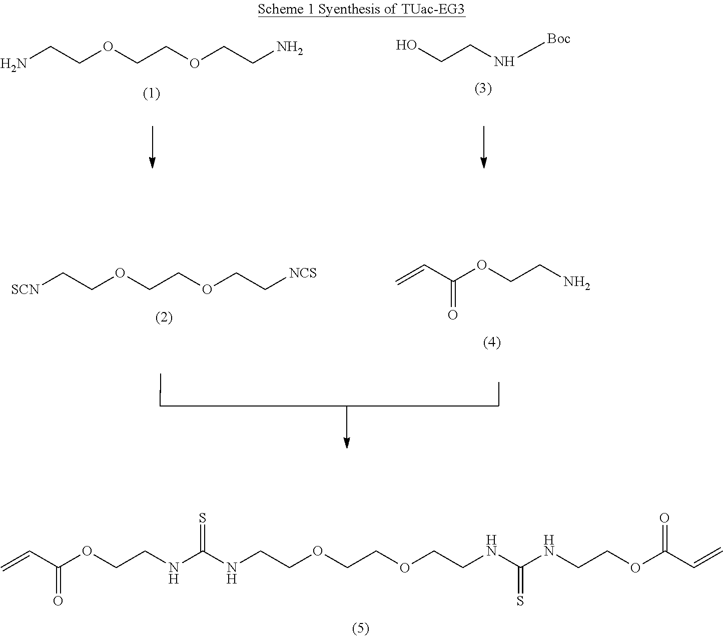

In a first step, triethylene glycol (1) having amino groups at both of its terminals and 2.0 equivalents of triethylamine in terms of molar ratio were added into a chloroform solvent, and the mixture was cooled to 0° C. Under an argon atmosphere, 2.0 equivalents of carbon disulfide was dropped into the reaction mixture. While the mixture was vigorously stirred, its temperature was returned to room temperature, followed by stirring for 2 hours. After that, the mixture was cooled to 0° C. again, and 2.0 equivalents of ethyl chloroformate was dropped thereinto, followed by vigorous stirring for 30 minutes. 30 Minutes thereafter, the temperature of the mixture was returned to room temperature, and the mixture was further stirred for 2 hours. The reaction mixture was washed with water and brine in the stated order, and was dried with sodium sulfate. After that, the solvent was evaporated. Next, the residue was thermally decomposed by evacuation at 120° C. for 2 hours. After the thermal decomposition, the decomposed product was purified with a column to provide NCS-EG3 (2).

A second step is the synthesis of an aminoethanol acrylate salt. Boc-aminoethanol (3) was dissolved in a methylene chloride solvent. Under an argon atmosphere, 2.0 equivalents of acryloyl chloride dissolved in methylene chloride was slowly dropped into the reaction mixture at 0° C., and the whole was vigorously stirred at 0° C. for 2 hours and at room temperature for 1 hour. After the reaction, the mixture was washed with saturated sodium hydrogen carbonate, and was dried with sodium sulfate. After that, the solvent was evaporated, and the residue was purified with a column to provide Boc-aminoethanol acrylate serving as an intermediate. The resultant intermediate was dissolved in a methylene chloride solvent, and under an argon atmosphere, 16 equivalents of trifluoroacetic acid was added to the solution at 0° C., and the mixture was stirred for 24 hours. After the reaction, the mixture was dried in a vacuum to provide a pasty aminoethanol acrylate salt (4).

In the final step, 6.0 equivalents of the aminoethanol acrylate salt (4) was dissolved in tetrahydrofuran. Under an argon atmosphere, 12 equivalents of triethylamine was added to the solution at 0° C., and the mixture was stirred for 5 minutes. After that, NCS-EG3 (2) was added to the mixture, and the whole was stirred for 3 hours. After the completion of the reaction, the solvent was evaporated, and the residue was purified with a column to provide TUac-EG3 (5) serving as a target.

### Production Example 2: Synthesis of T-Cy2

T-Cy2 was synthesized as a monomer of Production Example 2 through a route represented by the following scheme 2.

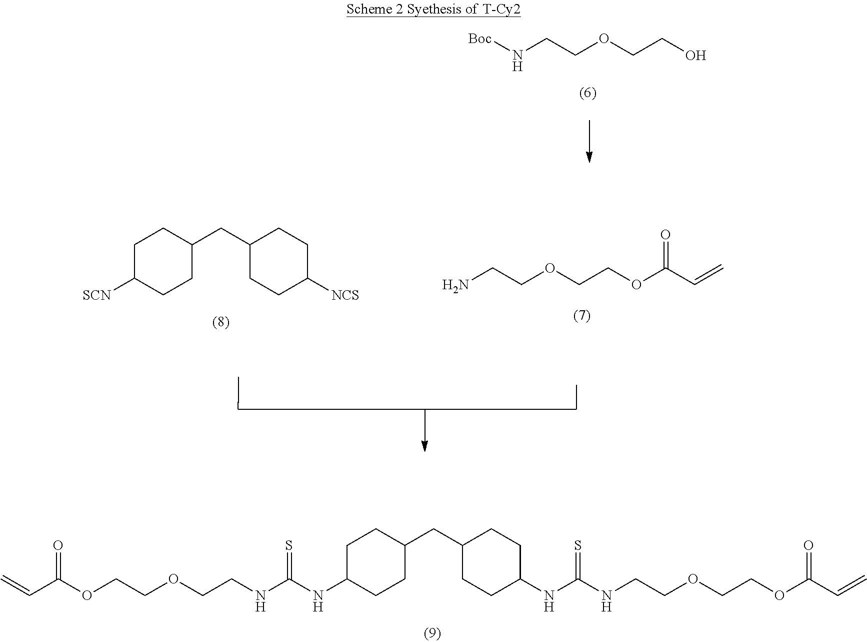

A first step is the synthesis of an aminoethylene glycol acrylate salt. Boc-aminoethylene glycol (6) was dissolved in a methylene chloride solvent. Under an argon atmosphere, 2.0 equivalents of acryloyl chloride dissolved in methylene chloride was slowly dropped into the reaction mixture at 0° C., and the whole was vigorously stirred at 0° C. for 2 hours and at room temperature for 1 hour. After the reaction, the mixture was washed with saturated sodium hydrogen carbonate, and was dried with sodium sulfate. After that, the solvent was evaporated, and the residue was purified with a column to provide Boc-aminoethylene glycol acrylate serving as an intermediate. The resultant intermediate was dissolved in a methylene chloride solvent, and under an argon atmosphere, 16 equivalents of trifluoroacetic acid was added to the solution at 0° C., and the mixture was stirred for 24 hours. After the reaction, the mixture was dried in a vacuum to provide a pasty aminoethylene glycol acrylate salt (7).

In a second step, 6.0 equivalents of the aminoethylene glycol acrylate salt was dissolved in tetrahydrofuran. Under an argon atmosphere, 12 equivalents of triethylamine was added to the solution at 0° C., and the mixture was stirred for 5 minutes. After that, commercial NCS-Cy2 (8) was added to the mixture, and the whole was stirred for 24 hours. After the completion of the reaction, the solvent was evaporated, and the residue was purified with a column to provide T-Cy2 (9).

### Production Example 3: Synthesis of Uac-EG3

Uac-EG3 was synthesized as a monomer of Production Example 3 bp the following scheme 3.

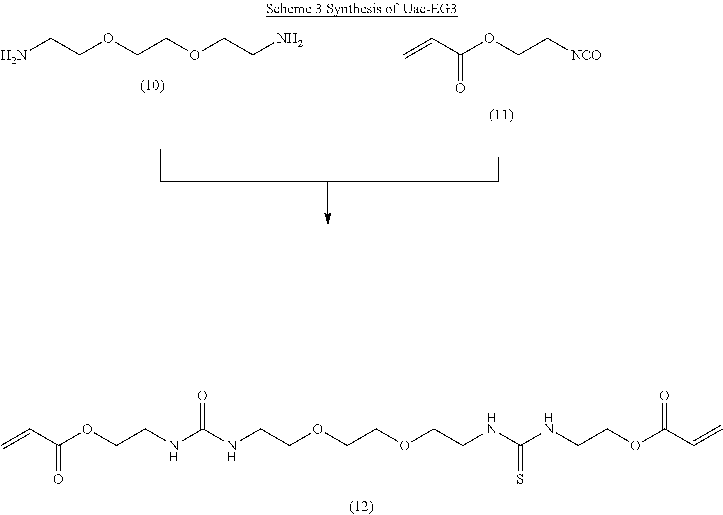

1,8-Diamino-3,6-dioxaoctane (10) was dissolved in a tetrahydrofuran solution, and under argon, the solution was cooled to 0° C. 2.8 Equivalents of isocyanatoethyl acrylate (11) was dropped into the solution, and the mixture was stirred at room temperature for 30 minutes. After the stirring, the reaction solution was dropped into diethyl ether that was vigorously stirred. Thus, a white precipitate was obtained. The precipitate was recovered by filtration, and was dried in a vacuum for 24 hours to provide Uac-EG3 (12) as a white solid.

### Production Example 4: Synthesis of U-Cy2

U-Cy2 was synthesized as a monomer of Production Example 4 through a route represented by the following scheme 4.

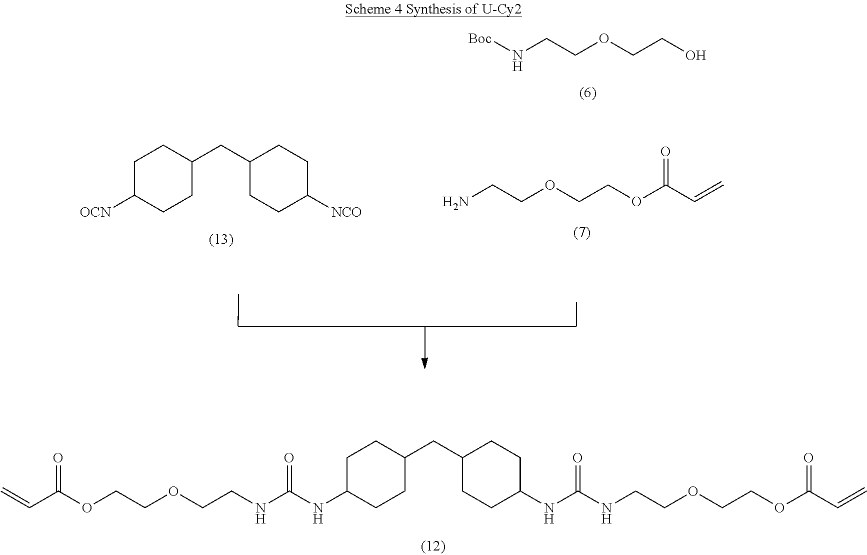

3.0 Equivalents of the aminoethylene glycol acrylate salt synthesized in Production Example 2 was dissolved in a tetrahydrofuran solution. Under an argon atmosphere, 6.0 equivalents of triethylamine was added to the solution at 0° C., and the mixture was stirred for 5 minutes. After that, commercial NCO-Cy2 (13) was added to the mixture, and the whole was stirred for 24 hours. After the completion of the reaction, the solvent was evaporated, and the residue was purified with a column to provide U-Cy2 (14).

### [Identification of Synthesized Monomer]

The chemical structures of the monomers synthesized in Production Examples 1 to 4 were identified by using ¹H NMR or ¹³C NMR. A solution obtained by dissolving 20 mg of a monomer sample in 0.7 mL of chloroform-d or deuterium oxide-d2 was used in each measurement. All the measurements were performed at room temperature.

### NMR

### TUac-EG3

¹H NMR (500 MHz, CDCl₃-d, δ): 7.18 (br 1H), 6.95 (br, 1H), 6.45 (d, J=17.2 Hz, 2H), 6.15 (dd, J=17.5, 10.6 Hz, 2H), 5.88 (d, J=10.9 Hz, 2H), 4.35 (t, J=5.2 Hz, 4H), 3.90-3.62 (m, 16H) ¹³C NMR (126 MHz, CDCl₃-d, δ): 182.9, 166.6, 131.8, 128.1, 70.1, 70.0, 63.3, 44.4, 44.1

### T-Cy2

¹H NMR (500 MHz, CDCl₃-d, δ): 6.51-6.42 (m, 5H), 6.16 (dd, J=17.5, 10.6 Hz, 2H), 5.89-5.87 (m, 2H), 4.33-3.65 (m, 18H), 2.10-0.95 (m, 20H)

¹³C NMR (126 MHz, CDCl₃-d, δ): 181.3, 166.2, 131.6, 128.1, 70.7, 69.2, 63.5, 63.4, 53.7, 44.5, 44.0, 33.7, 32.8, 31.9, 31.4, 29.3, 28.2

### Uac-EG3

¹H NMR (500 MHz, D₂O-d2, δ): 6.33 (d, J=17.2 Hz, 2H), 6.10 (dd, J=17.5, 10.6 Hz, 2H), 5.89 (d, J=10.3 Hz, 2H), 4.14 (t, J=5.4 Hz, 4H), 3.56-3.47 (m, 8H), 3.34 (t, J=5.2 Hz, 4H), 3.20 (t, J=5.2 Hz, 4H)

¹³C NMR (126 MHz, D₂O-d2, δ): 168.6, 160.5, 132.5, 127.5, 69.8, 69.6, 64.3, 39.5, 38.8

### U-Cy2

¹H NMR (500 MHz, CDCl₃-d, δ): 6.43 (dd, J=17.5, 1.4 Hz, 2H), 6.15 (dd, J=17.0, 10.5 Hz, 2H), 5.86 (d, J=10.3 Hz, 2H), 5.16 (br, 4H), 4.30 (s, 4H), 3.80-3.34 (m, 14H), 1.95-0.94 (m, 20H)

¹³C NMR (126 MHz, CDCl₃-d, δ): 166.3, 158.3, 131.4, 128.2, 71.2, 69.0, 63.7, 63.6, 49.6, 40.4, 33.8, 32.2, 30.1, 28.2

### Production Example 5: Synthesis of TUac-C8

TUac-C8 was synthesized as a monomer of Production Example 5 through a route represented by the following scheme 5.

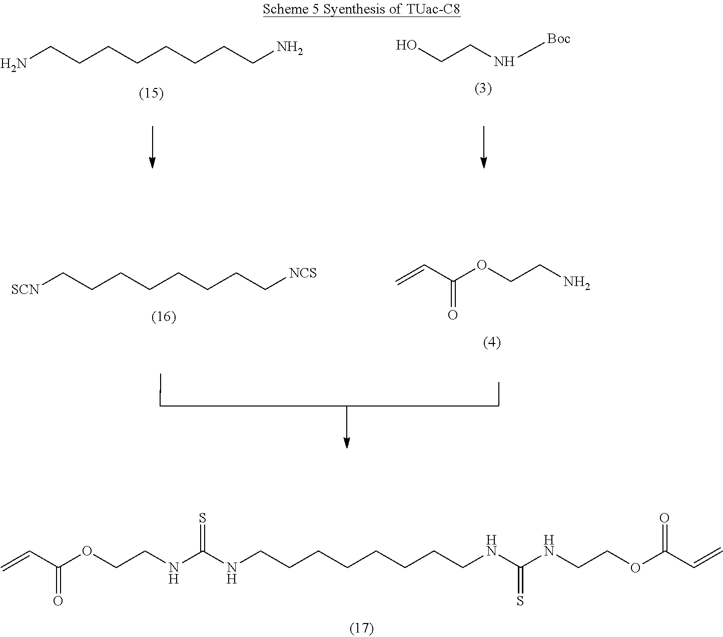

In a first step, 1,8-diaminooctane (15) was added to 2.0 equivalents of an aqueous solution of sodium hydroxide in terms of molar ratio, and the mixture was cooled to 0° C. Under an argon atmosphere, 2.0 equivalents of carbon disulfide was dropped into the reaction mixture. While the mixture was vigorously stirred, its temperature was returned to room temperature, followed by stirring for 2 hours. After that, the mixture was cooled to 0° C. again, and 2.0 equivalents of ethyl chloroformate was dropped thereinto, followed by vigorous stirring for 30 minutes. 30 Minutes thereafter, the temperature of the mixture was returned to room temperature, and the mixture was further stirred for 2 hours. The reaction mixture was extracted with chloroform, was washed with brine, and was dried with sodium sulfate. After that, the solvent was evaporated. Next, the residue was thermally decomposed by evacuation at 120° C. for 2 hours. After the thermal decomposition, the decomposed product was purified with a column to provide NCS-C8 (16).

6.0 Equivalents of the aminoethanol acrylate salt (4) synthesized in Production Example 1 was dissolved in a tetrahydrofuran solution. Under an argon atmosphere, 12 equivalents of triethylamine was added to the solution at 0° C., and the mixture was stirred for 5 minutes. After that, NCS-C8 (16) was added to the mixture, and the whole was stirred for 24 hours. After the completion of the reaction, the solvent was evaporated, and the residue was purified with a column to provide TUac-C8 (17).

### Production Example 6: Synthesis of TUac-Bn

TUac-Bn was synthesized as a monomer of Production Example 6 through a route represented by the following scheme 6.

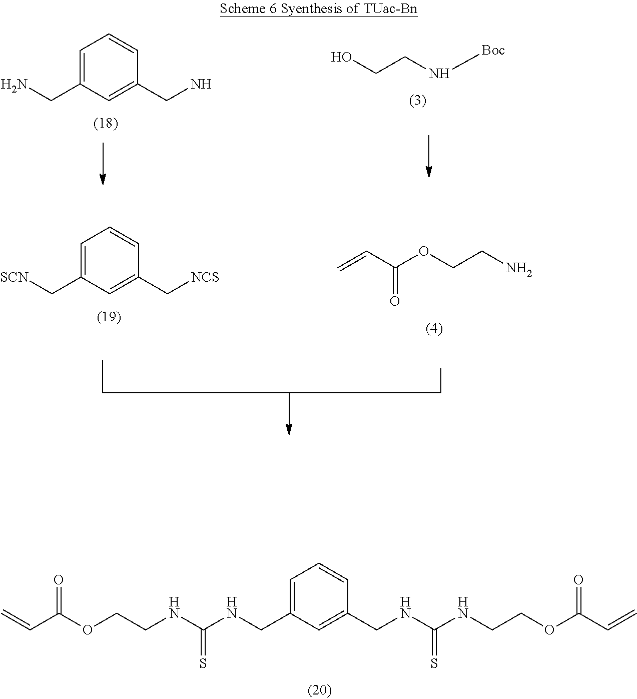

In a first step, xylylene diamine (18) and 2.0 equivalents of triethylamine in terms of molar ratio were loaded into a tetrahydrofuran solvent, and the mixture was cooled to 0° C. Under an argon atmosphere, 2.0 equivalents of carbon disulfide was dropped into the reaction mixture. While the mixture was vigorously stirred, its temperature was returned to room temperature, followed by stirring for 2 hours. After that, the mixture was cooled to 0° C. again, and 2.0 equivalents of ethyl chloroformate was dropped thereinto, followed by vigorous stirring for 30 minutes. 30 Minutes thereafter, the temperature of the mixture was returned to room temperature, and the mixture was further stirred for 2 hours. The reaction mixture was washed with water and brine in the stated order, and was dried with sodium sulfate. After that, the solvent was evaporated. Next, the residue was thermally decomposed by evacuation at 120° C. for 2 hours. After the thermal decomposition, the decomposed product was purified with a column to provide NCS-Bn (19).

6.0 Equivalents of the aminoethanol acrylate salt (4) synthesized in Production Example 1 was dissolved in a tetrahydrofuran solution. Under an argon atmosphere, 12 equivalents of triethylamine was added to the solution at 0° C., and the mixture was stirred for 5 minutes. After that, NCS-Bn (19) was added to the mixture, and the whole was stirred for 24 hours. After the completion of the reaction, the solvent was evaporated, and the residue was purified with a column to provide TUac-Bn (20).

### [Identification of Synthesized Monomer]

The chemical structures of the monomers synthesized in Production Examples 5 and 6 were identified by using ¹H NMR or ¹³C NMR. A solution obtained by dissolving 20 mg of a monomer sample in 0.7 mL of chloroform-d was used in each measurement. All the measurements were performed at room temperature.

### NMR

### TUac-C8

¹H NMR (500 MHz, CDCl₃-d, δ): 6.55-6.43 (m, 6H), 6.14 (dd, J=17.2, 10.3 Hz, 2H), 5.90 (dd, J=10.6, 1.4 Hz, 2H), 4.36 (t, J=5.2 Hz, 4H), 3.86 (s, 4H), 3.38 (s, 4H), 1.61-1.56 (m, 4H), 1.34 (d, J=17.2 Hz, 9H)

¹³C NMR (126 MHz, CDCl₃-d, δ): 182.0, 166.8, 132.0, 127.9, 63.2, 44.2, 44.1, 28.8, 28.7, 26.6

### TUac-Bn

¹H NMR (500 MHz, CDCl₃-d, δ): 7.64 (br, 3H), 7.30 (t, J=8.0 Hz, 1H), 7.16-7.10 (m, 3H), 6.45 (dd, J=17.2, 1.1 Hz, 2H), 6.14 (dd, J=17.2, 10.3 Hz, 2H), 5.87 (dd, J=10.6, 1.4 Hz, 2H), 4.93 (s, 4H), 3.92 (s, 4H), 3.18 (s, 4H)

¹³C NMR (126 MHz, CDCl₃-d, δ): 183.1, 165.9, 138.6, 131.7, 129.1, 128.0, 126.7, 121.5, 62.8, 48.3, 43.1

### Production Example 7: Tripropylene Glycol Diacrylate (TPA)

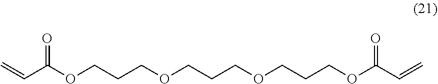 [SMILES: C=CC(=O)OCCCOCCCOCCCOC(=O)C=C]

A commercial product was used as tripropylene glycol diacrylate (TPA) (21).

### Production Example 8: Synthesis of T3EG

In the synthesis of T3EG, triethylene glycol having amino groups at both of its terminals and 0.95 equivalent of thiocarbonyldiimidazole in terms of molar ratio were used as monomers, and dimethylformamide was used as a solvent (under an argon atmosphere, the monomers were caused to react with each other at 80° C. for 6 hours). The dropping of the reaction mixture into methanol that was vigorously stirred provided pasty insoluble matter. The insoluble matter and a supernatant were separated from each other by decantation, and the insoluble matter was dissolved in chloroform. The solution was dropped into methanol again, and the resultant precipitate was separated. The dropping and separating operations were further repeated twice. The resultant pasty insoluble matter was dried in a vacuum at 80° C. for 24 hours to provide T3EG (22) as a yellowish transparent resin.

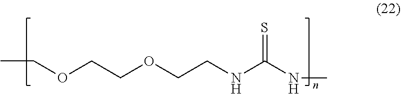

### [Identification of Synthesized Monomer]

The chemical structure of T3EG was identified by using ¹H NMR or ¹³C NMR. A solution obtained by dissolving 20 mg of a polymer sample in 0.7 mL of dimethylsulfoxide-d6 was used in each measurement. All the measurements were performed at room temperature.

¹H NMR (500 MHz, DMSO-d6, δ): 2.78 (br, CH₂NH₂), 3.45-3.59 (br, (S)NHCH₂), 7.49 (br, C(S)NH), ¹³C NMR (500 MHz, DMSO-d6, δ): 44.03, 69.054, 70.11, 183.34

The polymerization degree and number-average molecular weight of the polymer were determined from an integration ratio between a peak derived from a methylene adjacent to a terminal amino group and a peak derived from NH in ¹H NMR. A polymerization degree expected from a monomer loading ratio was 50. The weight-average molecular weight of T3EG determined by a Zimm-plot using multi-angle light scattering measurement was 9,500.

### Production Example 9: Synthesis of T-EG3

T-EG3 was synthesized as a monomer of Production Example 9 through a route represented by the following scheme 7.

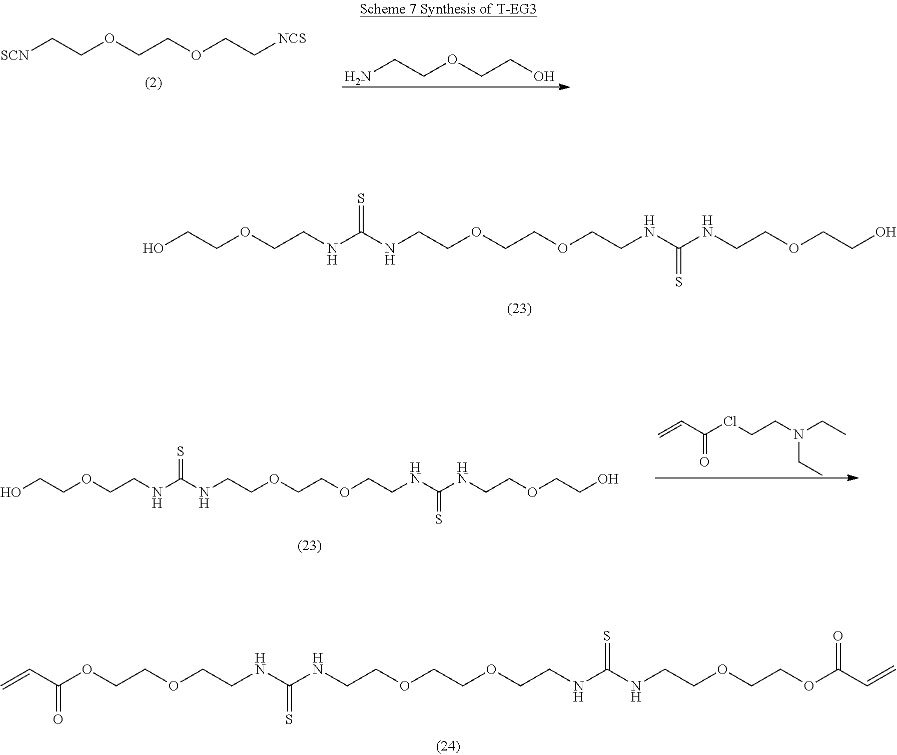

In a first step, NCS-EG3 (2) produced by a method in a previous report was added to a tetrahydrofuran solvent, and 4.0 equivalents of 2-(2-aminoethoxy)ethanol was dropped into the mixture, followed by stirring at room temperature for about 2 hours. After that, the reaction mixture was concentrated, and was purified with a column to provide di-OH-EG3 (23).

A second step is the synthesis of T-EG3. di-OH-EG3 (23) thus obtained was dissolved in a tetrahydrofuran solvent, and 4 equivalents of triethylamine was added to the solution. After that, under an argon atmosphere, 3 equivalents of acryloyl chloride was slowly dropped into the reaction mixture at 0° C., and the whole was vigorously stirred at −40° C. for 3 hours. After the reaction, the solvent was evaporated, and the residue was purified with a column to provide T-EG3 (24) serving as a target product.

### Production Example 10: Synthesis of T-C8

T-C8 was synthesized as a monomer of Production Example 10 through a route represented by the following scheme 8.

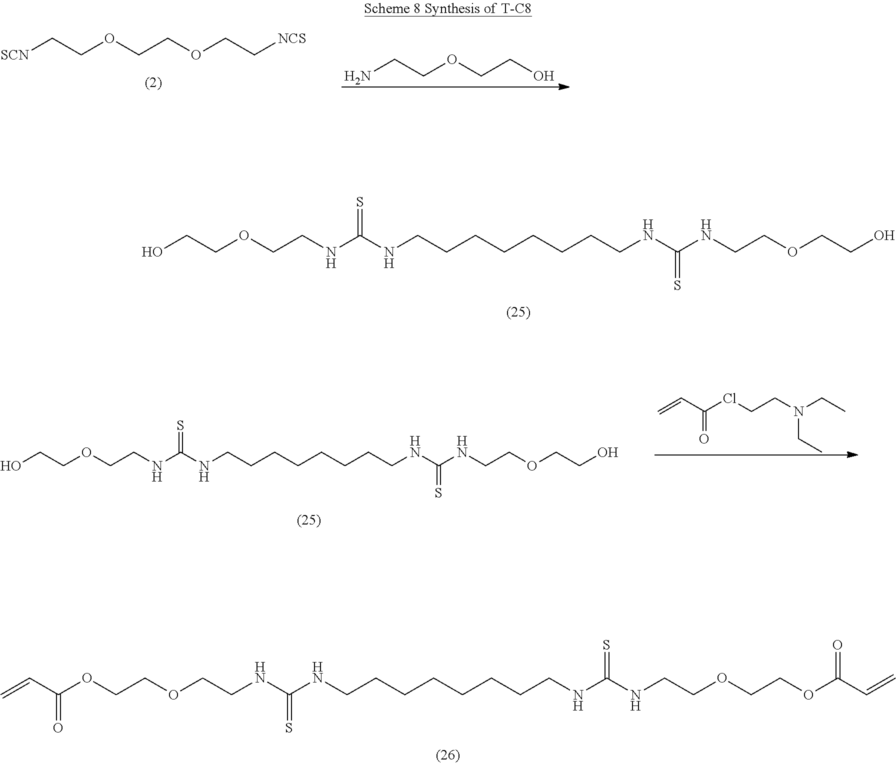

In a first step, NCS-C8 (16) produced by a method in a previous report was added to a tetrahydrofuran solvent, and 4.0 equivalents of 2-aminoethylethanol was dropped into the mixture at 0° C. Under that state, the temperature of the mixture was returned to room temperature, followed by stirring for about 15 hours. After that, the reaction mixture was concentrated, and was purified with a column to provide di-OH—C8 (25).

A second step is the synthesis of T-C8. di-OH—C8 (25) thus obtained was dissolved in a tetrahydrofuran solvent, and 5 equivalents of triethylamine was added to the solution. After that, under an argon atmosphere, 4 equivalents of acryloyl chloride was slowly dropped into the reaction mixture at 0° C., and the whole was vigorously stirred at −40° C. for 3 hours. After the reaction, the solvent was evaporated, and the residue was purified with a column to provide T-C8 (26) serving as a target product.

### [Identification of Synthesized Monomer]

The chemical structures of the monomers synthesized in Production Examples 9 and 10 were identified by using ¹H NMR or ¹³C NMR. A solution obtained by dissolving 20 mg of a monomer sample in 0.7 mL of chloroform-d1 was used in each measurement. All the measurements were performed at room temperature.

### NMR

di-OH-EG3

¹H NMR (500 MHz, DMSO-d6, δ): 7.52 (s, 6H), 4.57 (t, J=5.3 Hz, 2H), 3.54-3.42 (m, 28H)

¹³C NMR (151 MHz, DMSO-d6, δ): 183.1, 72.6, 70.1, 69.5, 69.4, 60.7, 44.1

### T-EG3

¹H NMR (600 MHz, CDCl₃-d1, δ): 7.05 (d, J=121.0 Hz, 2H), 6.44 (d, J=17.6 Hz, 2H), 6.18-6.13 (m, 2H), 5.87 (s, 2H), 4.33 (t, J=4.6 Hz, 4H), 3.75-3.61 (m, 24H)

¹³C NMR (151 MHz, CDCl₃-d1, δ): 182.6, 166.1, 131.4, 128.1, 70.0, 68.9, 67.9, 67.2, 63.4, 53.5, 44.6-di-OH—C8

¹H NMR (600 MHz, DMSO-d6, δ): 7.39 (d, J=92.4 Hz, 4H), 4.59 (t, J=5.3 Hz, 2H), 3.52-3.34 (m, 20H), 1.46 (t, J=6.1 Hz, 4H), 1.27 (s, 8H)

¹³C NMR (151 MHz, DMSO-d6, δ): 183.5, 72.6, 69.4, 60.7, 44.0, 29.2, 26.9

### T-C8

¹H NMR (600 MHz, CDCl₃-d1, δ): 6.46 (d, J=17.6 Hz, 2H), 6.29-6.15 (m, 3H), 5.90 (d, J=10.3 Hz, 2H), 4.35 (t, J=4.0 Hz, 4H), 3.76-3.42 (m, 16H), 1.63-1.58 (m, 6H), 1.35 (s, 8H)

¹³C NMR (151 MHz, CDCl₃-d1, δ): 182.3, 166.2, 131.5, 128.1, 70.2, 69.2, 63.1, 44.7, 41.5, 30.6, 28.8, 26.5

### Test Example 1. Test for Underwater Adhesive Property Between Two Targets Made of Different Materials

### (Method and Material)

To recognize the underwater adhesive property of a synthesized molecule, such an operation A as described below was entirely performed in deionized water.

Herein, a glass substrate is used as a first target.

Glass (Glass), stainless steel (Steel), wood (Wood), aluminum (Al), polypropylene (PP), or polytetrafluoroethylene (PTFE) was used as a second target.

### (Operation A):

The TUac-EG3 monomer synthesized in Production Example 1 was mounted on the second target, and a glass spacer having a diameter of 10 μm was mixed thereinto, followed by the overlapping of the first target so that the thickness of the monomer became 10 μm. The resultant was irradiated with light having a wavelength of 365 nm for 10 seconds. The operation was performed at 20° C. During the photoirradiation process, the monomer was polymerized and cured into a polymer to completely bond the two targets to each other. Thus, such an adhesion test piece that the targets were bonded to each other under a state in which the polymer was sandwiched therebetween was obtained.

Such bonding that all of its processes, that is, the application of the adhesive, the sandwiching, and the curing were performed in water as described above was defined as all-underwater bonding.

An overlap tensile shear test was used in the quantitative evaluation of an adhesive strength. The test method is known. A stress-strain curve was obtained by pulling the test piece at a rate of 1 mm/min. The adhesive strength (MPa) of the test piece was calculated by dividing a stress (N) at the time point when the breakage thereof occurred by the contact area (mm²) thereof.

### (Result)

TUac-EG3 showed underwater adhesive properties to all the targets. TUac-EG3 showed a stronger underwater adhesive property to a material having high surface energy, such as stainless steel, aluminum, or wood, out of those samples (FIG. 2A and FIG. 2B).

### Test Example 2. Test for Dependence of Adhesive Strength on Process

### (Method and Material)

To evaluate the dependence of an adhesive strength on a process, the adhesive strengths of samples produced through the following three processes were measured.
D (dry adhesion): The same operation as the operation A in the test for an underwater adhesive property of Test Example 1 was performed under a dry state. An adhesion test piece produced as follows was prepared: light was applied under the dry state and under a state in which TUac-EG3 was sandwiched between targets. A tensile test was performed by using the test piece at room temperature.
W (underwater adhesion): The same operation as the operation A in the test for an underwater adhesive property was performed in water. An adhesion test piece produced as follows was prepared: light was applied in the water and under a state in which TUac-EG3 was sandwiched between targets. A tensile test was performed by using the test piece at room temperature.
S (seawater adhesion): The same operation as the operation A in the test for an underwater adhesive property was performed in seawater. An adhesion test piece produced as follows was prepared: light was applied in the seawater and under a state in which TUac-EG3 was sandwiched between materials. A tensile test was performed by using the test piece at room temperature.

In addition, whether or not the bonded state was able to be maintained in the seawater when a weight of 100 g was applied to the sample obtained by bonding the targets in the seawater was recognized.

Further, the same experiment was performed by using a commercial epoxy-based underwater adhesive (manufactured by Cemedine Co., Ltd., product name: “UNDERWATER EPOXY”) for comparison.

### (Result)

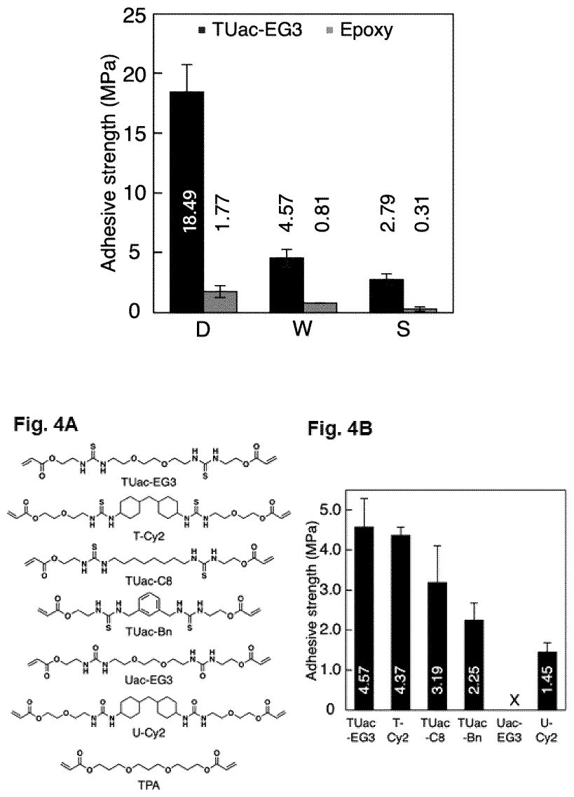

The results of the test for the dependence of an adhesive strength on a process are collectively shown in FIG. 3. It was found from the results that in each of the processes D, W, and S, TUac-EG3 showed an adhesive strength larger than that of the commercial epoxy-based adhesive, and that TUac-EG3 showed a high adhesive strength even in the seawater assumed in a practical scene such as the repair of a ship bottom. Further, it was recognized that the bonded state was able to be maintained in the seawater even 1 month after the test (data not shown).

### Test Example 3: Test for Underwater Adhesive Property

### (Method and Material)

A test for an underwater adhesive property based on the operation A of Test Example 1 was performed by using each of monomers illustrated in FIG. 4A.

### (Result)

All of thiourea monomers (TUac-EG3, T-Cy2, TUac-C8, and TUac-Bn) showed underwater adhesive properties. However, TUac-EG3 and T-Cy2 had underwater adhesive strength comparable to each other, and showed particularly high values out of thiourea adhesives (FIG. 4B). In addition, Uac-EG3 was not able to be used in underwater bonding because of the following reason: Uac-EG3 was a water-soluble white solid, and hence when an attempt was made to perform the underwater bonding, Uac-EG3 was dissolved in water. Although U-Cy2 showed an underwater adhesive property, T-Cy2 showed an underwater adhesive property three or more times as strong as the foregoing. In addition, TPA was water-insoluble, and was hence able to be cured in water, but did not show any underwater adhesive property.

### Test Example 4. Evaluation of Persistence of Adhesive Strength

### (Method and Material)

The operation A was performed in water by the same procedure as that of the test for an underwater adhesive property of Test Example 1, and microscope slides bonded to each other under a state in which TUac-EG3 and T-Cy2 were each sandwiched therebetween were prepared and used as a sample. Each of the prepared samples was left to stand still in deionized water at room temperature for 24 hours, and was then evaluated for its adhesive strength.

### (Result)

Both of TUac-EG3 and T-Cy2 showed high underwater adhesive strengths even 24 hours after the still standing (FIG. 5). In addition, it was found that while the adhesive strength of TUac-EG3 reduced by about 50% 24 hours thereafter, the adhesive strength of T-Cy2 was able to be maintained at as high as 80% or more of the initial value. Accordingly, it was found that T-Cy2 had more excellent durability.

### Test Example 5. Swelling Test

### (Method and Material)

TUac-EG3 and T3EG in JP 6574259 B2 were evaluated for their water absorption behaviors. Specifically, first, each of the polymers was synthesized under a dry state. After that, the polymer was immersed in deionized water, and its mass was measured every specific time. Then, a mass change (%) was calculated by: dividing a difference between the masses before and after the immersion by the mass before the immersion; and multiplying the quotient by 100. The mass change was defined as a water absorption amount, and was used as an indicator of an evaluation of water-absorption behaviors.

### (Result)

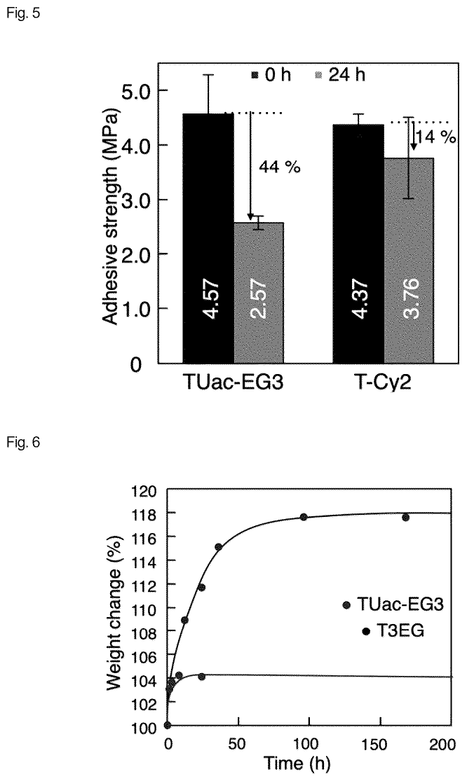

It was found that while the TUac-EG3 of the present invention swelled by only 4%, T3EG swelled by as large as 20% (FIG. 6). Accordingly, the TUac-EG3 of the present invention has overcome problems with moisture absorption and water absorption behaviors. The fact that the problem with the water absorption behaviors has been overcome has enabled the TUac-EG3 to maintain its adhesive strength over a long time period. It is conceivable that T-Cy2 having a cyclohexyl structure is further suppressed from swelling.

### Test Example 6. Test for Adhesive Property

### (Method and Material)

To recognize the underwater adhesive property of a two-component adhesive without use of any light, such an operation B as described below was entirely performed in deionized water.

Herein, a first material and a second material may be identical to or different from each other in kind. In this Test Example, glass plates were used as the first target and the second target. In addition, the two liquids of the adhesive were named a liquid A and a liquid B, and were each defined as described below.
(Liquid A): a thiourea adhesive including one of TUac-EG3 or T-Cy2
(Liquid B): a mixed solution obtained by mixing a branched thiol (one of Tri-SH (27) or Hexa-SH (28)) and an amine catalyst (hexylamine) (29)

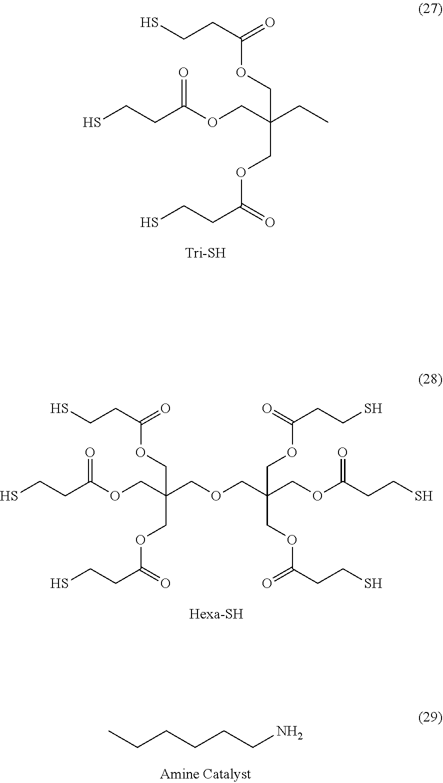

### (Operation B):

A mixture obtained by mixing the liquid A and the liquid B so that the amounts of C═C and SH were equal to each other was applied on the second target, and a glass spacer having a diameter of 10 μm was mixed thereinto. The first target was overlapped thereon so that the thickness of the mixture became 10 μm, followed by fixation with a clip. The operation was performed at 20° C. After that, the resultant was left to stand in water for a certain time period. Thus, a thiol-ene reaction occurred to form and cure a three-dimensional network. Thus, such an adhesion test piece that the targets were bonded to each other under a state in which the polymer was sandwiched therebetween was obtained.

### (Result)

It was found that the polymer showed underwater adhesive properties in all the combinations of TUac-EG3/Tri-SH, TUac-EG3/Hexa-SH, T-Cy2/Tri-SH, and T-Cy2/Hexa-SH. As described above, the use of a two-component adhesive enabled underwater bonding even when no light was used. The general-purpose properties of a thiourea acrylate adhesive such as the bonding of opaque materials were able to be extended.

### Test Example 7. Dependence of Underwater Adhesive Property on Type of Cross-linker

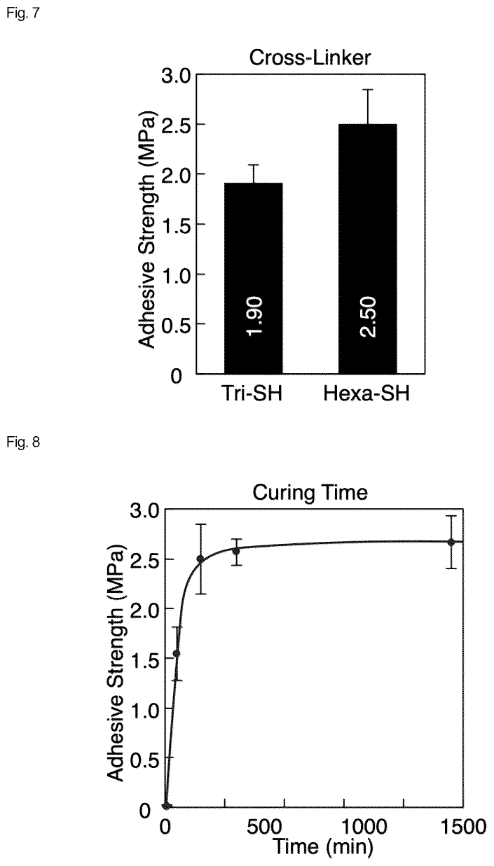

The dependence of an underwater adhesive property on the type of a cross-linker was examined. Each of Tri-SH (27) and Hexa-SH (28) was used as the cross-linker, and was mixed in an amount of 1 equivalent with respect to the C═C of the T-Cy2 monomer, followed by the production of an adhesion test piece in accordance with the method of Test Example 6. As a result of an adhesion test in accordance with the tensile shear test of Test Example 1 (however, a tensile rate was set to 5 mm/min), it was found that when Hexa-SH was used, a high underwater adhesive property was obtained as compared to that when Tri-SH was used (FIG. 7).

### Test Example 8. Dependence on Curing Time

An underwater adhesive property at each elapsed time from the mixing of T-Cy2 and the cross-linker Hexa-SH was examined. As a result of an adhesion test in accordance with the tensile shear test of Test Example 1 (however, a tensile rate was set to 5 mm/min), it was found that the adhesive strength of the mixture converged to a substantially constant value at an elapsed time of about 3 hours (FIG. 8).

### Test Example 9. Dependence of Underwater Adhesive Property on Ratio Between Two Liquids

An underwater adhesive property when the equivalent amount of each of the branched thiol (Hexa-SH), which was a two-component mixed cross-linker, and the amine catalyst (hexylamine) with respect to a monomer was changed was examined. Each adhesion test piece was produced in accordance with the method of Test Example 6. As a result of an adhesion test in accordance with the tensile shear test of Test Example 1 (however, a tensile rate was set to 5 mm/min), it was found that Hexa-SH showed the highest underwater adhesive property when an equivalent ratio between Hexa-SH and the C═C of the monomer was 1:1. In addition, it was found that when the ratio of hexylamine was 50 mol % with respect to the C═C of the monomer, its underwater adhesive strength showed a local maximum point, and hence the ratio was most suitable (Table 1).

TABLE 1
Dependence of underwater adhesive property
on ratio of cross-linker or catalyst
Cross-Linker Ratio

| C═C/SH | Amine (eq) | Adhesion |
|---|---|---|
| 1/3 | 0.5 | 1.0 |
| 2/3 | 0.5 | 1.3 |
| 1/1 | 0.5 | 2.5 |
| 3/2 | 0.5 | 1.8 |
| 3/1 | 0.5 | 1.3 |
| 1/1 | 0.0 | 0.03 |
| 1/1 | 0.25 | 0.7 |
| 1/1 | 0.75 | 2.1 |
| 1/1 | 1.0 | 2.0 |

### Test Example 10. Dependence of Underwater Adhesive Property on Monomer

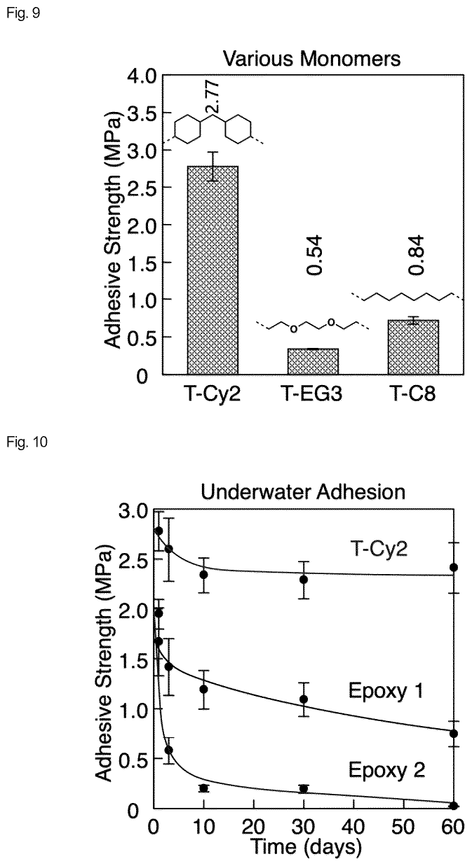

Herein, a two-component mixed adhesive was used as an adhesive, and an adhesion test piece was produced in accordance with the method of Test Example 6. T-Cy2, T-EG3, and T-C8 were each used as the monomer of the (liquid A). A mixed solution of Hexa-SH and hexylamine was used as the (liquid B). As a result of an adhesion test in accordance with the tensile shear test of Test Example 1 (however, a tensile rate was set to 5 mm/min), each of the adhesives showed an underwater adhesive property, and the adhesive produced by using T-Cy2 showed the highest underwater adhesive property (FIG. 9).

### Test Example 11. Test for Dependence of Underwater Adhesive Property on Time

To evaluate the durability of adhesion in water, the following test was performed. Herein, a two-component mixed adhesive was used as an adhesive, and an adhesion test piece was produced in accordance with the method of Test Example 6. T-Cy2 was used as the monomer of the liquid A. The liquid B of Test Example 6 was used as the (liquid B). The prepared sample was left to stand still in deionized water at room temperature for 1 day, 3 days, 10 days, or 30 days, and then a tensile test was performed in accordance with the tensile shear test of Test Example 1 (however, a tensile rate was set to 5 mm/min). The results are shown in FIG. 10. It was found from the foregoing results that the underwater adhesive of the present invention maintained a sufficient adhesive strength even after the lapse of a long time period in the water. In addition, it was found that the adhesive forces of two kinds of epoxy adhesives produced by using commercial two-component mixed adhesives reduced with the lapse of time, and the two-component mixed adhesive produced by using T-Cy2 (hereinafter also simply referred to as “two-component mixed T-Cy2”) had the highest adhesive strength and was excellent in durability. Further, the two-component mixed T-Cy2 showed a strong and durable adhesive property even in seawater (FIG. 11).

### Test Example 12. Dependence on Targets

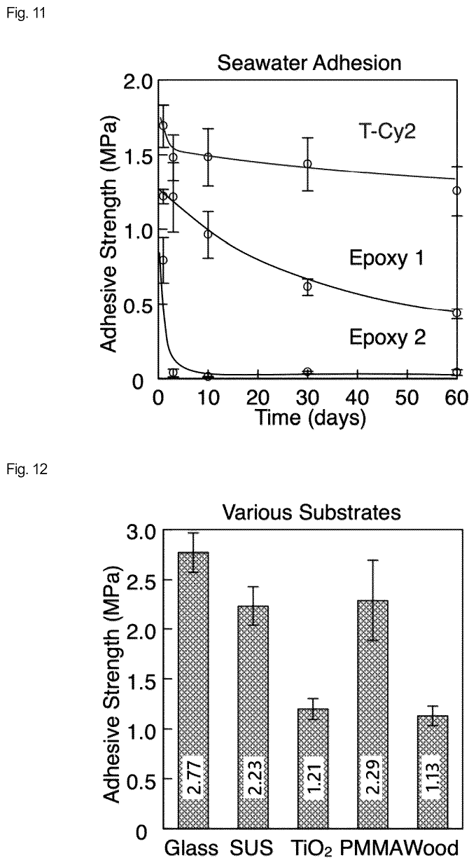

The two-component mixed T-Cy2 also shows an adhesive property to a material except glass. To recognize the dependence of adhesion on a base material, an adhesion test piece was produced by using targets made of various materials instead of two sheets of glass in accordance with the method of Test Example 6, and was examined for its underwater adhesive strength by a tensile test under the same conditions as those of the tensile shear test of Test Example 1 (however, a tensile rate was set to 5 mm/min). The two-component mixed T-Cy2 showed a stronger underwater adhesive property to a target having high surface energy, such as stainless steel (SUS stainless-steel plate), titanium oxide, wood, or PMMA (FIG. 12).

### Test Example 13. Optical Instant Adhesive Property of Two-Component Mixed Adhesive

The inventors have conceived of the use of light for shortening the curing time of a two-component mixed adhesive. Herein, liquids A and C are mixed. The same liquid as that of Test Example 6 was used as the liquid A, and the liquid C was newly produced as described below. (Liquid C): a mixed solution obtained by mixing a branched thiol (Hexa-SH (28)) and a commercial photopolymerization initiator (2,2-dimethoxy-2-phenylacetophenone, Tokyo Chemical Industry Co., Ltd.)

Glass substrates were used as a first target and a second target. A mixture obtained by mixing the liquid A and the liquid C so that the amounts of C═C and SH were equal to each other was mounted on the second target, and the first target was overlapped thereon, followed by fixation with a clip. The operation was performed at 20° C. After that, light was applied, and a thiol-ene reaction occurred to form and cure a three-dimensional network. Thus, such an adhesion test piece that the targets were bonded to each other under a state in which the polymer was sandwiched therebetween was obtained.

### (Result)

It was found that T-Cy2/Hexa-SH showed an underwater adhesive strength comparable to that in the case where no light was used (Test Example 7). However, a time period required for the curing of the adhesive was only 1 minute. As described above, even a two-component mixed adhesive can be rapidly cured as required.

1. A compound, which is represented by the following formula (1): 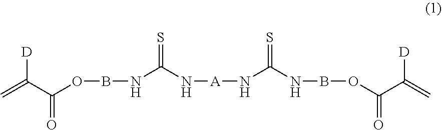
in the formula (1), A and B satisfy a relationship (I) or (II):
(I) one of A or B represents a group represented by the following formula (2): 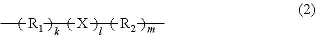
in the formula (2),
R₁ and R₂ each independently represent a substituted or unsubstituted alkylene group having 1 to 16 carbon atoms,
X is selected from the following groups: 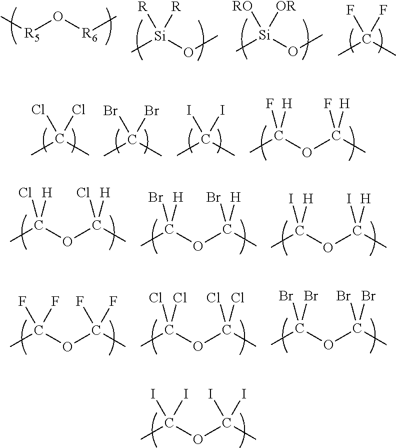
R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “1” represents an integer of 1 or more, and “m” represents an integer of 0 or more,
another of A or B represents one group selected from the following (a) to (c), or has a structure in which the following two or more groups (b) are linked to each other, or a structure in which the following two groups (c) are linked to each other:
(a) a substituted or unsubstituted alkylene group having 1 to 16 carbon atoms;
(b): 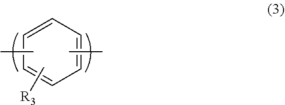
when one bonding moiety of main chain bonding moieties is defined as a 1-position, another bonding moiety thereof may be any one of a 2-position, a 3-position, and a 4-position, and R₃ represents a hydrogen atom, or 1 to 4 monovalent, identical or different, and substituted or unsubstituted alkyl groups each having 1 to 6 carbon atoms; and
(c): 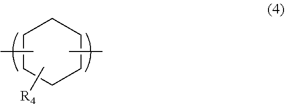
when one bonding moiety of main chain bonding moieties is defined as a 1-position, another bonding moiety thereof may be any one of a 2-position, a 3-position, and a 4-position, and R₄ represents a hydrogen atom, or 1 to 4 monovalent, identical or different, and substituted or unsubstituted alkyl groups each having 1 to 6 carbon atoms, and
when the other of A or B has the structure in which the following two or more groups (b) are linked to each other, or the structure in which the following two groups (c) are linked to each other, a linking group is —O— or —C(Rₐ)(R_{b})—, and Rₐ and R_{b} each independently represent hydrogen or an alkyl group having 1 to 6 carbon atoms; and
(II) both of A and B each independently represent a group represented by the following formula (2): 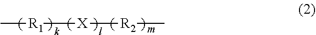
in the formula (2),
R₁ and R₂ each independently represent a substituted or unsubstituted alkylene group having 1 to 16 carbon atoms,
X is selected from the following groups: 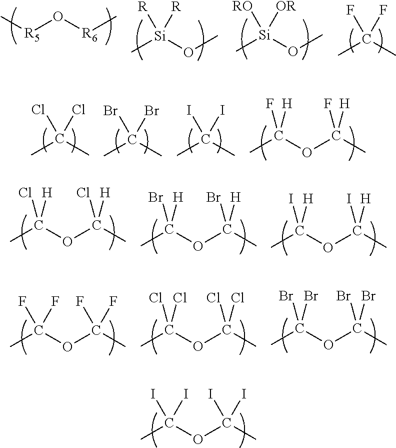
R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “1” represents an integer of 1 or more, and “m” represents an integer of 0 or more, and
D represents hydrogen or a methyl group.

2. The compound according to claim 1, wherein the compound is obtained by reacting a compound represented by the following formula (9) with a compound represented by the following formula (10): 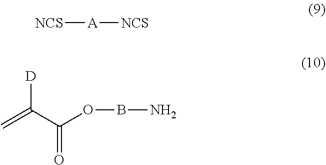
A in the formula (9) and B in the formula (10) satisfy a relationship (I) or (II):
(I) one of A or B represents a group represented by the following formula (2): 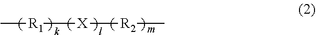
in the formula (2),
R¹ and R₂ each independently represent a substituted or unsubstituted alkylene group having 1 to 16 carbon atoms,
X is selected from the following groups: 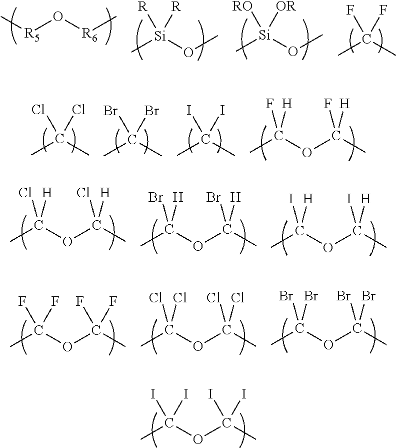
R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “1” represents an integer of 1 or more, and “m” represents an integer of 0 or more,
another of A or B represents one group selected from the following (a) to (c), or has a structure in which the following two or more groups (b) are linked to each other, or a structure in which the following two groups (c) are linked to each other:
(a) a substituted or unsubstituted alkylene group having 1 to 16 carbon atoms;
(b): 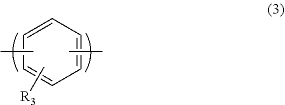
when one bonding moiety of main chain bonding moieties is defined as a 1-position, another bonding moiety thereof may be any one of a 2-position, a 3-position, and a 4-position, and R₃ represents a hydrogen atom, or 1 to 4 monovalent, identical or different, and substituted or unsubstituted alkyl groups each having 1 to 6 carbon atoms; and
(c): 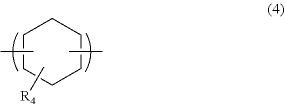
when one bonding moiety of main chain bonding moieties is defined as a 1-position, another bonding moiety thereof may be any one of a 2-position, a 3-position, and a 4-position, and R₄ represents a hydrogen atom, or 1 to 4 monovalent, identical or different, and substituted or unsubstituted alkyl groups each having 1 to 6 carbon atoms, and
when the other of A or B has the structure in which the following two or more groups (b) are linked to each other, or the structure in which the following two groups (c) are linked to each other, a linking group is —O— or —C(Rₐ)(R_{b})—, and Rₐ and R_{b} each independently represent hydrogen or an alkyl group having 1 to 6 carbon atoms; and
(II) both of A and B each independently represent a group represented by the following formula (2): 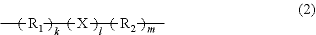
in the formula (2),
R₁ and R₂ each independently represent a substituted or unsubstituted alkylene group having 1 to 16 carbon atoms,
X is selected from the following groups: 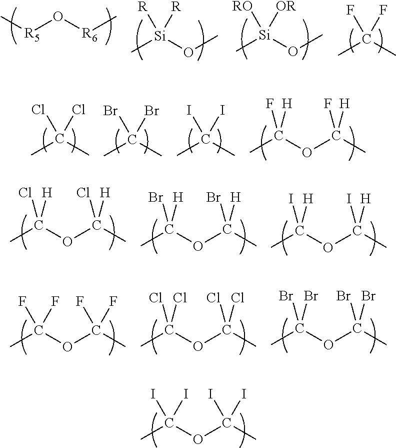
R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “1” represents an integer of 1 or more, and “m” represents an integer of 0 or more, and
D represents hydrogen or a methyl group.

3. The compound according to claim 1, wherein the group represented by the formula (2) is a group represented by the following formula (2a): 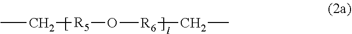
where R₅ and R₆ each independently represent a substituted or unsubstituted alkylene group having 1 to 16 carbon atoms, and a substituent of each of R₅ and R₆ is a linear or branched alkyl group having 1 to 6 carbon atoms, a cyclohexyl group, a phenyl group, or a halogen, and
“1” represents an integer of from 1 to 5.

4. The compound according to claim 1,
wherein A represents a group represented by the formula (2a): 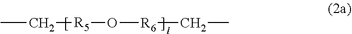
where R₅ and R₆ each independently represent a substituted or unsubstituted alkylene group having 1 to 16 carbon atoms, and a substituent of each of R₅ and R₆ is a linear or branched alkyl group having 1 to 6 carbon atoms, a cyclohexyl group, a phenyl group, or a halogen, and
“1” represents an integer of from 1 to 5, and
wherein B represents (a) the substituted or unsubstituted alkylene group having 1 to 16 carbon atoms.

5. The compound according to claim 1,
wherein A has the structure in which the two groups (c) are linked to each other, and
wherein B represents a group represented by the formula (2a): 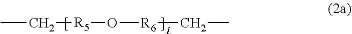
where R₅ and R₆ each independently represent a substituted or unsubstituted alkylene group having 1 to 16 carbon atoms, and a substituent of each of R₅ and R₆ is a linear or branched alkyl group having 1 to 6 carbon atoms, a cyclohexyl group, a phenyl group, or a halogen, and
“1” represents an integer of from 1 to 5.

6. A polymer, which is obtained by polymerizing the compound of claim 1.

7. A polymer, which is represented by the following formula (11): 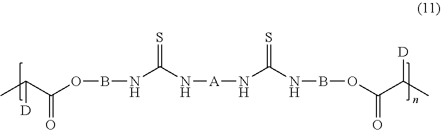
in the formula (11), A and B satisfy a relationship (I) or (II):
(I) one of A or B represents a group represented by the following formula (2): 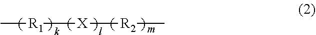
in the formula (2),
R₁ and R₂ each independently represent a substituted or unsubstituted alkylene group having 1 to 16 carbon atoms,
X is selected from the following groups: 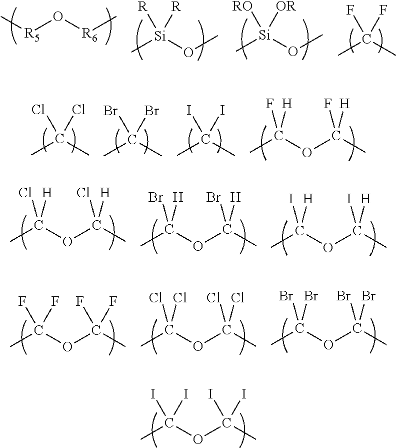
R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “1” represents an integer of 1 or more, and “m” represents an integer of 0 or more,
another of A or B represents one group selected from the following (a) to (c), or has a structure in which the following two or more groups (b) are linked to each other, or a structure in which the following two groups (c) are linked to each other:
(a) a substituted or unsubstituted alkylene group having 1 to 16 carbon atoms;
(b): 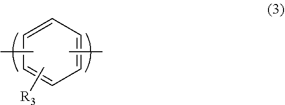
when one bonding moiety of main chain bonding moieties is defined as a 1-position, another bonding moiety thereof may be any one of a 2-position, a 3-position, and a 4-position, and R₃ represents a hydrogen atom, or 1 to 4 monovalent, identical or different, and substituted or unsubstituted alkyl groups each having 1 to 6 carbon atoms; and
(c): 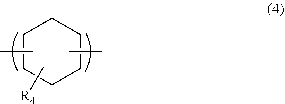
when one bonding moiety of main chain bonding moieties is defined as a 1-position, another bonding moiety thereof may be any one of a 2-position, a 3-position, and a 4-position, and R₄ represents a hydrogen atom, or 1 to 4 monovalent, identical or different, and substituted or unsubstituted alkyl groups each having 1 to 6 carbon atoms, and
when the other of A or B has the structure in which the following two or more groups (b) are linked to each other, or the structure in which the following two groups (c) are linked to each other, a linking group is —O— or —C(Rₐ)(R_{b})—, and Rₐ and R_{b} each independently represent hydrogen or an alkyl group having 1 to 6 carbon atoms; and
(II) both of A and B each independently represent a group represented by the following formula (2): 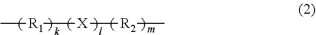
in the formula (2),
R₁ and R₂ each independently represent a substituted or unsubstituted alkylene group having 1 to 16 carbon atoms,
X is selected from the following groups: 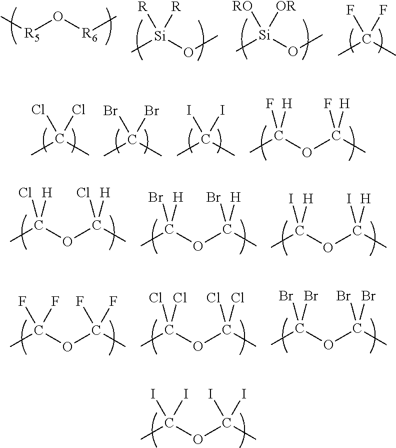
R₅ and R₆ each independently represent a substituted or unsubstituted alkylene group having 1 to 16 carbon atoms,
R represents a substituted or unsubstituted alkylene group having 1 to 16 carbon atoms, and
“k” represents an integer of 0 or more, “1” represents an integer of 1 or more, and “m” represents an integer of 0 or more,
D represents hydrogen or a methyl group, and
“n” represents a polymerization degree of a repeating unit.

8. An adhesive comprising the compound according to claim 1.

9. An adhesive comprising:
the compound of claim 1; and
a compound having two or more thiol groups.

10. A bonding method comprising:
applying an adhesive including the compound of claim 1 to a surface of a target; and
curing the applied adhesive by irradiating light to the adhesive.

11. The bonding method according to claim 10,
wherein the target is a first target,
wherein the bonding method further comprises bringing a second target into contact with the adhesive after the step of applying the adhesive so that the adhesive is arranged between the first target and the second target, and
wherein the applied adhesive is cured through the application of the light to the adhesive after the step of bringing the second target into contact with the adhesive.

12. A bonding method comprising:
mixing the compound of claim 1 and a compound having two or more thiol groups; and
applying the mixture to a surface of a target, followed by curing of the mixture.

13. The bonding method according to claim 11, wherein the curing step is performed in water, in an aqueous solution, in an aqueous suspension, or in a high-humidity environment.

14. The bonding method according to claim 10, wherein the target is a material selected from the group consisting of: a metal, glass, wood and a synthetic resin.

15. A use of the compound of claim 1 for production of an adhesive.

16. An adhesive comprising the polymer according to claim 6.
